(19) United States
(12) Patent Application Publication
(10) Pub. No.: US 2024/0118267 A1
(43) Pub. Date: Apr. 11, 2024

(54) CELL CULTURE GROWTH APPARATUS AND METHOD OF UNDERSIDE ATTACHMENT TO A SURFACE USING CELL SEEDING

(71) Applicants: Hannah VIOLA, Atlanta, GA (US); Jocelyn R. GRUNWELL, Atlanta, GA (US); Shuichi TAKAYMA, Atlanta, GA (US); Rabindra TIROUVANZIAM, Atlanta, GA (US); Georgia Tech Research Corporation, Atlanta, GA (US); Children's Healthcare of Atlanta, Inc., Atlanta, GA (US); Emory University, Atlanta, GA (US)

(72) Inventors: Hannah Viola, Atlanta, GA (US); Jocelyn R. Grunwell, Atlanta, GA (US); Shuichi Takayama, Atlanta (GA); Rabindra Tirouvanziam, Atlanta, GA (US)

(21) Appl. No.: 18/280,393

(22) PCT Filed: Mar. 3, 2022
(86) PCT No.: PCT/US2022/018810

§ 371 Date: Sep. 5, 2023

Related U.S. Application Data
(60) Provisional application No. 63/156,068, filed on Mar. 3, 2021.

(51) Int. Cl. G01N 33/50; C12M 1/32; C12N 5/00; C12N 5/071

(52) U.S. Cl. CPC G01N 33/5029; C12M 23/12; C12N 5/0018; C12N 5/0688; G01N 33/5044; C12N 2500/34; G01N 2800/12

(57) ABSTRACT

High-throughput co-culture models, for example, relevant to the cell barrier interface, are disclosed that employ under-side cell seeding in a miniaturized and automated system and process. The seeding method, which can be implemented in a scalable and low-cost manner, can eliminate the need for an inversion process by adjusting/optimizing the density of the cell suspension medium to float cells of interest so they can attach under a membrane.

### CROSS-REFERENCE TO RELATED APPLICATIONS

This application claims priority to, and the benefit of, U.S. Provisional Application No. 63/156,068, filed on Mar. 3, 2021, which is expressly incorporated herein by reference in its entirety.

### STATEMENT OF GOVERNMENT INTEREST

This invention was made with government support under grant no. R01HL136141 awarded by the National Institutes of Health (NIH). The government has certain rights in the invention.

### TECHNICAL FIELD

This disclosure relates to a method and system for cell culture growth, in particular cell culture growth via underside attachment to a growth platform using cell seeding operations, e.g., to screen/evaluate agents and/or study disease.

### BACKGROUND

High-throughput tissue barrier cell cultures (as “tissue culture models”) are employed in the research and development of new drugs and scientific research to provide critical insights on how the barrier function can respond to therapeutics, pathogens, and toxins. However, such models often emphasize multiplexing capability at the expense of physiologic relevance. Particularly, the airway-vascular barrier is typically modeled with epithelial cell monoculture, but this neglects the substantial contribution of endothelial cell feedback in the coordination of barrier function.

There is a benefit for systems or apparatuses that can grow more physiologically relevant high-throughput tissue barriers and associated methods, e.g., for screening/evaluating agents or studying disease.

### SUMMARY

In accordance with the purposes of the disclosed materials and methods, as embodied and broadly described herein, the disclosed subject matter, in various aspects, relates to compounds, compositions, systems, tools, and apparatuses, and associated methods of making and/or using said compounds, compositions, systems, tools, and apparatuses.

High-throughput co-culture models, for example, relevant to the epithelium/endothelium interface, are disclosed that employ underside cell seeding in a miniaturized and automated system and process. The seeding method, which can be implemented in a scalable and low-cost manner, can eliminate the need for an inversion process by adjusting/optimizing the density of the cell suspension medium to float cells of interest so they can attach under a membrane.

In one example, a system comprising a multi-well model of a small airway-vascular barrier can be implemented that employs serum-free, glucocorticoid-free air-liquid differentiation. The system can be employed to grow a polarized epithelial-endothelial co-culture having a mature barrier function, appropriate intercellular junction staining, and epithelial-to-endothelial transmission of inflammatory stimuli such as poly(I:C). Further, the system can be used to expose the polarized epithelial-endothelial co-culture, for example, to influenza A virus (PR8 strain) and human beta-coronavirus (OC43 strain), to initiate a dose-dependent inflammatory response that can propagate from the epithelium cells located in an underside culture to the endothelium cell located on a top-side culture. While this tissue model has application in the evaluation of the air-blood barrier, the exemplary underside seeding method and system can also be employed in the preparation of various co-culture tissue barrier models for scalable, physiologic screening.

Disclosed herein is a method of seeding cells to an underside of a surface; the method comprising: a) providing a surface, wherein said surface is upright and not inverted; b) providing cells, wherein the cells are located on or attached to an underside of said surface when the surface is in an upright position; c) providing a cell suspension medium, wherein said cell suspension medium has a higher density than the cells, and d) contacting said surface with said cells and said cell suspension medium, wherein said cells float on a surface of said cell suspension medium, thereby seeding the underside of the surface with said cells.

The cell suspension medium can comprise at least one of: a polymer or mixture of polymers; a small molecule or mixture of small molecules; a synthetic or naturally derived particle or a mixture of particles; a coacervate; a colloid; a protein or mixture thereof; and a hydrogel or preparation thereof. The cell suspension medium can comprise a mixture of components of these components dissolved or otherwise suspended in a carrier solution selected from the group consisting of water, a buffer, a salt solution, and a cell culture medium. The cells used can be adherent cells, and the plate can be a multiple-well (for example, a 96-well plate or a 384-well plate or any multi-well plate disclosed herein). In some embodiments, the plate can be a Transwell plate having any number of wells described herein, such as a 96-well Transwell plate, a 384-well Transwell plate, or a 384-well pillar plate. The culture suspension can be denser than the cell by at least 0.01 g/mL. The cell suspension media can comprise a polymer, particle, or protein solution that has increased density relative to the cell. The cell suspension media can be a dextran solution or a density gradient medium solution, or a combination thereof. Air-liquid interface (ALI) culture can then be performed on the underside of said surface. The seeded cells can include the H441 club cell line, primary epithelial cells, or engineered epithelial cells, or a combination thereof. Different cell types can be seeded on the underside and topside of said surface.

Also disclosed herein is an in vitro tool, system, or apparatus that can be used in the screening and/or evaluation of active agents that can modulate a cell barrier (e.g., an epithelial barrier). The in vitro tool may include a porous substrate configurable to an upright position, an endothelial barrier, and an epithelial barrier, wherein the endothelial barrier is formed on a first surface of the substrate, wherein the epithelial barrier is formed on a second surface of the substrate, and wherein the second surface is located at an underside of the substrate. In some embodiments, wherein the substrate is placed in or is a part of a well of a multiple-well plate (e.g., a 96-well plate, a 384-well plate, or other plate disclosed herein). In some embodiments, the distance between the second surface of the substrate and the bottom of the well is configurable. In some examples, the distance between the second surface of the substrate and the bottom of the well is about 0.5 mm to 5 mm. In some examples, the distance between the second surface of the substrate and the bottom of the well is about 1.3 mm. In some embodiments, The bottom well has a diameter approximately 9 mm.

Also disclosed herein is an in vitro tool, system, or apparatus that can be used for the screening and/or evaluating for active agents that modulate an epithelial barrier, an epithelial-endothelial barrier, or cells placed upon the barrier, wherein said in vitro tool, system, or apparatus is prepared by any of the above-discussed methods. In some embodiments, the cells placed upon the barrer are immune cells or non-immune cells. Accordingly, in some aspects, disclosed herein is an in vitro tool, system, or apparatus that can be used for the screening and/or evaluating for active agents that modulate cells (e.g., immune cells or non-immune cells) placed upon an epithelial barrier or an epithelial-endothelial barrier, wherein said in vitro tool, system, or apparatus is prepared by any of the above-discussed method.

Further described is a method of using an in vitro tool, system, or apparatus that can be used to determine interactions with the in vitro tool comprising an epithelial-endothelial barrier and an input variable, wherein said method comprises exposing the in vitro tool to the input variable, placing suspended cells on the topside of the epithelial-endothelial barrier (e.g., on the endothelial side), and determining interactions between the input variable and the in vitro tool. The method can be performed using air-liquid interface (ALI) culture. The input variable can be introduced to the in vitro tool comprising an epithelial-endothelial barrier via an aerosol, vapor, gas, or a fluid. The tool's epithelial barrier can be on the underside of the well, and are therefore more easily exposed to the input variable than a traditional assay. The input variable can be a test compound, molecule, reagent, or organism. The organism can be a virus, such as PR8 or other influenza viruses, OC43 SARS-CoV-2, or other coronaviruses. Exposing the in vitro tool to an input variable can comprise dipping the underside of the in vitro tool into a plate with arrays of wells containing the input variable, then lifting the in vitro tool out from these wells and culturing said cells. The cell culturing can occur by transferring the in vitro tool to a plate with empty wells or other air culture methods, which exposes the cells to an air interface. The assay can be exposed to nanoparticles or pollution particles. Differential recovery of liquids and cells and cell-derived materials (e.g. RNA, DNA) can be obtained from each side of the culture membrane separately. The cells (e.g., suspension cells) of the assay can be immune cells, cancer cells, or other mammalia cells. During culturing, chemotaxis of the cells from a top side to the underside of the filter can occur. The method can be employed in a high throughput process.

Also disclosed is a dense cell culture media comprising a homogeneous mixture of about 50/50 v/v % cell culture media and a solution of 60% w/v iodixanol in water with a density of 1.32 g/mL. Accordingly, in some examples, the 50/50 v/v % solutions have a density of about 1.16 g/mL.

### DESCRIPTION OF DRAWINGS

The accompanying figures, which are incorporated in and constitute a part of this specification, illustrate several aspects described below.

FIGS. 1A-1B show automatable underside seeding enabled by density-driven flotation forms a robust monolayer of NCI-H441 in co-culture with endothelium. FIG. 1A) Seeding on the underside of Transwell membranes is typically accomplished by insert inversion (bottom panel). The density-driven flotation method enables upright underside seeding without inversion (top panel) for facile generation of bilayer co-cultures. Here, the utility of this method is demonstrated in a model of the air-blood barrier by coculturing epithelium with endothelium in 96-well Transwell inserts. Histology section with H&E stain (center, top panel) shows epithelial-endothelial culture on opposite sides of the Transwell membrane. Confocal imaging of the epithelium stained for F-actin (center, bottom panel) shows that H441 cells form a monolayer with our seeding method. FIG. 1B shows that, At initial seeding time (Day 0), H441 cells float to the underside of the Transwell membrane where they can adhere for 2 hours. By 24 hours (Day 1), the majority of epithelial cells are attached. On Day 8, following 6 days of air-liquid interface culture, H441 cells are limited to the culture area that is fed from the opposite side, and both layers are confluent. Scale bar, 20 μM (top 2 images), 3.13 mm (bottom).

FIGS. 2A-2C show that co-cultured NCI-H441 cells and HUVECs show polarization and differentiation leading to the formation of a robust air-blood barrier. FIG. 2A) Trans-epithelial electrical resistance (TEER) measures the strength of intercellular junctions. Co-cultured H441-HUVEC bilayers exhibit synergistically elevated TEER (p<0.05 compared to both monocultures for every day measured, Supplemental Data) compared to their respective monocultures during seven days of air-liquid interface culture. FIG. 2B) In four independent experiments, seeding efficiency was almost 97%, meaning that, on average, 93 of 96 wells met quality control criteria. Successful wells are defined as those meeting the criteria: TEER >286 Ω·cm2 on Day 6 of ALI culture; confluent H441 and HUVEC monolayers determined by visual inspection; retention of the medium in the top compartment without leakage, also determined by visual inspection. FIG. 2C) H441 cells in co-culture with HUVECs at an air-liquid interface develop appropriate cellular junctions indicating barrier formation. (Left, top) H441 cells demonstrate polarization of the epithelium by ZO-1 localization at the apical air-cell boundary (blue) with intercellular e-cadherin indicating the formation of adherens junction complexes (yellow). Scale bar, 10 μM. (Left, bottom) ZO-1 (blue) tight junction stain on co-cultured, ALI-differentiated H441 cells indicates robust intercellular barrier formation. Scale bar, 25 μM. (Center) E-cadherin staining in co-cultured H441 cells indicates robust intercellular barrier formation and epithelial monolayer. Scale bar, 50 μM. (Right) Co-cultured HUVECs express adherens junctional protein VE-cadherin, indicating the formation of an endothelial barrier. Scale bar, 25 μM.

FIG. 3 shows GFP-transduced NCI-H441 cells (green) and RFP-transduced HUVEC cells in co-culture. Day 0 shows floating seed (left) and after 2 hours of attachment (right). Day 5-Day 10 shows the progression to a culture area limited to that shared with the upper compartment due to air-liquid interface (ALI) culture conditions

FIG. 4 shows NCI-H441 cells grow into the membrane pores to contact the endothelium. Left, F-actin stain with phalloidin shows the endothelial layer with interspersed epithelial cell protrusions (arrows). The top cross-section shows the H441 layer on top, endothelial layer on the bottom, with protrusions extending between the layers (arrowhead). Right, GFP-expressing H441 cells in co-culture grow protrusions through the membrane pores (arrow). Images taken by spinning disc confocal microscopy.

FIG. 5A shows the box (gray) encloses the microplate (orange) on an elevated, flat surface above a surrounding water source (blue). The plate rests on a ridged surface with two tabs for easy removal. The box consists of two parts, a bottom chamber containing the water, and a lid. The left panel of FIG. 5B shows the top view showing that the water, in blue, surrounds the microplate. The right panel of FIG. 5B shows perpendicular cutout showing that the microplate is elevated above the water moat by a plastic support. FIG. 5C shows this view showing the humidity chamber without the lid, microplate, or water.

FIG. 6 shows an overview of the example application showing utility of the invention. The high throughput barrier can be exposed to toxins, aerosol, chemicals, or patient-derived specimen on the epithelial barrier side, in the presence or absence of test therapeutic(s). Following this, measurement of barrier function (TEER), chemoattractiveness (migrated neutrophil number), and drug efficacy interactions (dose-response and mixture curves) can be evaluated. (Bottom panel) The exemplary workflow incorporates human-like adverse reactions, and organ-level events in vitro in a high throughput, low-biological variability platform.

FIG. 7 shows Dose-response and donor differences in transmigration assay. Primary human neutrophils were transmigrated to chemoattractant leukotriene B4 (LTB4) or recombinant human protein IL-8 (HEK-expressed, activity tested). Primary human peripheral neutrophils from 3 healthy donors (F, 27 y; F, 25 y; F, 61 y) were placed on the endothelial compartment and allowed to transmigrate for 16 hours. Then, neutrophils were counted using the Cytoflex flow cytometer. n=2-3 technical replicates per condition. Two-way ANOVA for each chemoattractant separately showed effect of chemoattractant dose (p<0.0001) and donor (p<0.0001) on the number of counted neutrophils. Multiple comparisons with Tukey's post hoc t-test showed significant differences between dose for a given chemoattractant and donor. For all graphs: * p<0.05, ** p<0.01, *** p<0.001, **** p<0.0001. Statistics were performed in GraphPad Prism 9. Statistics not shown for bottom panel graphs.

Left panel of FIG. 8 shows that the epithelial cell side was treated with the ROS probe Carboxy-H2DCFDA (ThermoFisher, C400), then the epithelial side was exposed to different types of SOA suspensions at the concentrations designated in the graphs for 2 hours (n=4 per conditions). Production of reactive oxygen species (ROS) was measured through increase in mean fluorescence intensity (MFI). Two-way ANOVA showed no significant effect of SOA type on ROS production but significant effect of dose (p<0.0001). Multiple comparisons using post-hoc Tukey's t-test identified significant differences between SOA suspension concentrations. Right panel of FIG. 8 shows that the epithelial side was treated with SOA for 24 hours. Then, IL-8 produced on the endothelial side was measured by ELISA (R&D Systems). Two-way ANOVA showed effect of both SOA type (p<0.0001) and dose (p<0.0001) on IL-8 production. Multiple comparisons with Tukey's post hoc t-test showed significant differences between dose for a given SOA. For both graphs: * p<0.05, ** p<0.01, *** p<0.001, **** p<0.0001. Statistics were performed in GraphPad Prism 9.

FIG. 9 shows Serum incorporation into transmigration assay. Primary human neutrophils were transmigrated to chemoattractant leukotriene B4 (LTB4) at 300 uM with or without the presence of human serum. Pooled human male AB+, complement preserved serum was diluted into culture medium on the endothelial (top), epithelial (bottom), or both compartments of the Transwell chamber. The bottom compartment contained 300 uM LTB4. Primary human peripheral neutrophils from one donor (F, 27 y) were placed on the endothelial compartment and allowed to transmigrate for 16 hours. Then, neutrophils were counted using the Cytoflex flow cytometer. n=8 technical replicates per condition. The number of transmigrated neutrophils significantly increased with the addition of 2-6% vol/vol serum in the top compartment. Serum in the bottom compartment induced aggregation of neutrophils by observation, inhibiting effective cell counting and suggesting serum in the bottom compartment is not viable for transmigration assays. Two-way ANOVA showed effect of both serum dose (p<0.0001) and serum placement (top/bottom/both) (p<0.0001) on the number of counted neutrophils. Multiple comparisons with Tukey's post hoc t-test showed significant differences between dose for a given serum placement. For both graphs: * p<0.05, ** p<0.01, *** p<0.001, **** p<0.0001. Statistics were performed in GraphPad Prism 9.

FIG. 10 shows that pre-treatment of neutrophils with Baricitinib dose-dependently inhibits transmigration. Images show neutrophils collected in the bottom well after transmigration to either TNF-a (top) or LTB4 (bottom. Large cells are contaminating epithelial cells. 4×EVOS Brightfield with phase contrast.

FIG. 11 shows assay schematic using primary patient-derived samples to test drugs. Top: Primary peripheral human neutrophils from healthy donors are isolated from whole blood and treated with a test drug. Bottom: Tracheal aspirate is collected and processed from critically ill patients with lung disease. Assay (right): Neutrophils are added to the top well and patient sample to the bottom well. Wells with and without drug are compared to test effect of drugs on the number of migrated neutrophils.

Left panel of FIG. 12 show that the endothelial cell side was treated with DNA-histone microwebs for 24 hours (n=3 replicates per condition). Microwebs were either composed of 1:1 histone:DNA or 3:1 histone:DNA (mass:mass). Percent of initial TEER was measured with the EVOM-2 voltmeter (World Precision Instruments). Two-way ANOVA across all 4 groups showed significant effect of exposure (DNA vs. histone vs. MW 1:1 vs. MW 1:3) (p<0.0001) and significant effect of exposure dose (p<0.0001). Multiple comparisons with Tukey's post hoc t-test showed significant differences between dose for a given exposure. For both graphs: * p<0.05, ** p<0.01, *** p<0.001, **** p<0.0001. Statistics were performed in GraphPad Prism 9,

FIG. 13 shows transmigration to CF sputum. Primary peripheral blood neutrophils were transmigrated through an epithelial-endothelial bilayer to pooled CF airway supernatant derived from sputum and diluted with culture media at 1:1, 1:2, 1:6 and 1:12 for 18 hours. i) Flow cytometry showed that compared to peripheral blood neutrophils, transmigrated neutrophils lost CD62L (L-selectin) and became activated. Neutrophils almost entirely lost expression of CD16, a hallmark of CF neutrophil activation. They also had increased CD66b and CD63 indicative of activation and degranulation. ii) Further, the number of recruited neutrophils was dependent on concentration of sputum, similar to the cytokine results. iii) Incubation with CF sputum on the epithelium for 18 hours during the transmigration also resulted in upregulation of endothelial e-selectin on the high (1:1) concentration of sputum but not low concentration (1:12).

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the n^{th} 10 reference in the list. All references cited and discussed in this specification are incorporated herein by reference in their entireties and to the same extent as if each reference was individually incorporated by reference.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list.

### Terminology

Terms used throughout this application are to be construed with ordinary and typical meaning to those of ordinary skill in the art. However, Applicant desires that the following terms be given the particular definition as defined below.

An exemplary method is disclosed that employs density-driven cell buoyancy to facilitate underside attachment without inversion. As an initial demonstration of this underside seeding method, a study was conducted that constructed a co-culture model of the small airways (bronchioles). This region of the lung, which is close to the alveoli, is heavily involved in the mediation of inflammatory responses during toxin and pathogen exposure. Successful barrier maintenance in this region is critical to prevent acute lung injury from developing after insults or infection. The study showed that upon stimulation of the epithelial side of the exemplary engineered air-blood barrier with bacterial or viral insults, the apposing endothelium exhibited prothrombotic (e.g., vWF release) and proinflammatory (e.g., IL-8 secretion) responses. The many wells available for testing conveniently allowed a comparison of the effect of different viruses, MOI, and time points.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

It must also be noted that, as used in the specification and the appended claims, the singular forms “a,” “an,” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

As used here, the term “active agent” is used herein to refer to a chemical compound, composition, or organism that has a biological effect. The terms also encompass pharmaceutically acceptable, pharmacologically active derivatives of active agents specifically mentioned herein, including, but not limited to, salts, esters, amides, prodrugs, active metabolites, isomers, fragments, analogs, organisms, and the like. When the term “active agent” is used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

“Administration” to a subject or “administering” includes any route of introducing or delivering to a subject an agent. Administration can be carried out by any suitable route, including intravenous, intraperitoneal, and the like. Administration includes self-administration and the administration by another.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history, can also be used.

Under the invention, the terms “cell culture” and “tissue culture” may be used interchangeably and denote the maintenance of cells in vitro, in suspension culture, in a liquid medium, or on a surface.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

Contacting: Placement in direct physical association, for example, solid, liquid, or gaseous forms. Contacting includes, for example, direct physical association of fully- and partially-solvated molecules.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Inhibit,” “inhibiting,” and “inhibition” mean to decrease an activity, response, condition, disease, or another biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down-regulate the activity of the described target protein. A control sample (untreated with inhibitors) can be assigned a relative activity value of 100%. Inhibition of a described target protein is achieved when the activity value relative to the control can be assigned about 80%, optionally 50% or 25, 10%, 5%, or 1%.

The term “isolating” as used herein refers to isolation from a biological sample, i.e., blood, plasma, tissues, exosomes, or cells. As used herein, the term “isolated,” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

The term “polymer” as used herein refers to a relatively high molecular weight organic compound, natural or synthetic, whose structure can be represented by a repeated small unit, the monomer. Synthetic polymers are typically formed by the addition or condensation polymerization of monomers. The polymers used or produced in the present invention are biodegradable. The polymer is suitable for use in the body of a subject, i.e., is biologically inert and physiologically acceptable, non-toxic, and is biodegradable in the environment of use, i.e., can be resorbed by the body. The term “polymer” encompasses all forms of polymers, including, but not limited to, natural polymers, synthetic polymers, homopolymers, heteropolymers or copolymers, addition polymers, etc.

As used herein, the term “primary cells” refers to cells that are freshly obtained from cells or tissue taken from an organism. The cells or tissue from which a primary culture is derived is termed an explant. “Primary cells” will grow for a variable but finite length of time in culture, after which time they senesce and eventually die. Under the embodiments of the invention, primary cultures can be derived from a variety of tissue sources, and a number of techniques for their isolation from human tissue are known in the art.

A “cell line” refers to a population of cells derived from a single explant which are characterized as having the potential for unlimited proliferation in vitro.

As discussed herein, a “subject” may be any applicable human, animal, or other organisms, living or dead, or other biological or molecular structure or chemical environment, and may relate to particular components of the subject, for instance, specific tissues or fluids of a subject (e.g., human tissue in a particular area of the body of a living subject), which may be in a particular location of the subject, referred to herein as an “area of interest” or a “region of interest.” It should be appreciated that, as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc. It should be appreciated that the subject may be any applicable human patient, for example.

The terms “treat,” “treating,” “treatment,” and grammatical variations thereof, as used herein, include partially or completely delaying, alleviating, mitigating, or reducing the intensity of one or more attendant symptoms of a disorder or condition and/or alleviating, mitigating or impeding one or more causes of a disorder or condition. Treatments according to the invention may be applied preventively, prophylactically, palliatively, or remedially. Prophylactic treatments are administered to a subject prior to onset (e.g., before obvious signs of a lung disorder), during early onset (e.g., upon initial signs and symptoms of a lung disorder), or after an established development of a disease (e.g., a lung disorder). Prophylactic administration can occur for several days to years prior to the manifestation of symptoms of a disease (e.g., a lung disorder).

“Therapeutically effective amount” or “therapeutically effective dose” of a composition (e.g., a composition comprising an active agent) refers to an amount that is effective to achieve a desired therapeutic result. In some embodiments, a desired therapeutic result is the control of a lung disorder or a symptom thereof. Therapeutically effective amounts of a given therapeutic agent will typically vary with respect to factors such as the type and severity of the disorder or disease being treated and the age, gender, and weight of the subject. The term can also refer to an amount of a therapeutic agent or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of the agent in the formulation, and the like), and a variety of other factors that are appreciated by those of ordinary skill in the art. In some instances, a desired biological or medical response is achieved following the administration of multiple dosages of the composition to the subject over a period of days, weeks, or years.

The following patents, applications, and publications as listed below and throughout this document are hereby incorporated by reference in their entirety herein.

### Methods of Seeding Cells

Challenges remain to seeding cells to an underside of a surface. In some aspects, disclosed herein is a method of seeding cells to an underside of a surface, said method comprising:
providing a surface, wherein said surface is in an upright position;
providing a mixture that comprises cells and a cell suspension medium, wherein said cell suspension medium has a higher density than the cells; and
contacting the mixture with an underside of said surface, thereby seeding the underside of the surface with said cells.

In some embodiments, the cell suspension medium comprises one or more of:
a polymer or mixture of polymers;
a small molecule or mixture of small molecules;
a synthetic or naturally derived particle or mixture of particles;
a coacervate;
a colloid;
a protein or mixture thereof; and
a hydrogel or preparation thereof.

In some embodiments, the cell suspension medium further comprises a carrier solution selected from the group consisting of water, a buffer, a salt solution, and a cell culture medium.

The cell suspension medium is denser than the cells but can be tolerated by the contacted cells. Accordingly, in some embodiments, the cell suspension medium is denser than the cell by at least about 0.001 g/mL, 0.002 g/mL, 0.004 g/mL, 0.008 g/mL, 0.01 g/mL, 0.02 g/mL, 0.04 g/mL, 0.05 g/mL, 0.06 g/mL, 0.07 g/mL, 0.08 g/mL, 0.09 g/mL, 0.1 g/mL, 0.2 g/mL, 0.3 g/mL, 0.4 g/mL, 0.5 g/mL, 0.6 g/mL, 0.7 g/mL, 0.8 g/mL, 0.9 g/mL, 1.0 g/mL, 1.2 g/mL, 1.4 g/mL, 1.6 g/mL, 1.8 g/mL, or 2.0 g/mL. In some embodiments, the cells' density is about 1.05 g/mL. In some embodiments, the density of the cell suspension medium is about 1.06 g/mL, 1.07 g/mL, 1.08 g/mL, 1.09 g/mL, 1.10 g/mL, 1.11 g/mL, 1.12 g/mL, 1.13 g/mL, 1.14 g/mL, 1.15 g/mL, 1.16 g/mL, 1.17 g/mL, 1.18 g/mL, 1.19 g/mL, 1.2 g/mL, 1.22 g/mL, 1.24 g/mL, 1.26 g/mL, 1.28 g/mL, 1.3 g/mL, 1.35 g/mL, 1.4 g/mL, 1.45 g/mL, 1.5 g/mL, 1.55 g/mL, 1.6 g/mL, 1.65 g/mL, 1.7 g/mL, 1.8 g/mL, 1.9 g/mL, 2.0 g/mL, 2.2 g/mL, 2.3 g/mL, 2.4 g/mL, 2.5 g/mL, 2.6 g/mL, 2.7 g/mL, 2.8 g/mL, 2.9 g/mL, or 3.0 g/mL.

In some embodiments, the cells are attached cells. In some embodiments, the cells are epithelial cells. In some embodiments, the cells are on the surface of the suspension medium.

In some aspects, disclosed herein is a tool, system, and/or apparatus prepared by the method disclosed herein, wherein the tool, system, and/or apparatus comprises a surface comprising cells seeded to the underside of said surface, wherein said method comprises:
providing a surface, wherein said surface is in an upright position;
providing a mixture that comprises cells and a cell suspension medium, wherein said cell suspension medium has a higher density than the cells; and
contacting the mixture with an underside of said surface, thereby seeding the underside of the surface with said cells.

The term “adherent cells” herein refers to cells which can be attached to a surface to grow. In some embodiments, the cells are adherent cells. In some embodiments, the cells are epithelial cells or fibroblasts.

In some embodiments, the surface is placed in or is a part of a multiple-well pate (e.g., a 6-well plate, a 12-well plate, a 24-well plate, a 48-well plate, a 96-well plate, or a 384-well plate).

Also, in some aspects, disclosed herein is a method of creating a cell barrier, said method comprising:
providing a substrate in an upright position, wherein said substrate has a first surface and a second surface, and wherein the second surface is located at an underside of the substrate when said substrate is in an upright position;
providing a first mixture of cells that comprises a plurality of adherent cells and a cell suspension medium, wherein said cell suspension medium has a higher density than the adherent cells;
contacting the first mixture of cells with the second surface, thereby seeding the underside of the substrate with the adherent cells;
adding a second mixture of cells that comprises a plurality of cells (e.g., endothelial cells) and a cell culture medium on the first surface of the substrate;
removing the cell suspension medium to form a mixed cell culture having an air and liquid interface whereby the adherent cells seeded on the underside of the substrate are exposed to the air, and whereby the cells (e.g., endothelial cells) on the first surface of the substrate are in contact with a cell culture medium, and
culturing the cells at the air and liquid interface to form a barrier comprising the adherent cells.

In some embodiments, the cells are adherent cells. In some embodiments, the cells are epithelial cells or fibroblasts.

Also disclosed herein is a method of creating an epithelial-endothelial barrier. The term “epithelial-endothelial barrier” disclosed herein refers to a cell culture grown on a substrate comprising a first surface and a second surface, wherein the second surface comprises a plural of epithelial cells, and the first surface comprises a plural of endothelial cells. The first and second surfaces can be parallel or non-parallel to one another. In some embodiments, at least one of the first and second surfaces is a textured surface. In some embodiments, at least one of the first and second surfaces is an angled surface. In some embodiments, at least one of the first and second surfaces is a curved surface. In some examples, the substrate is porous between the first surface and the second surface. In some embodiments, the substrate is a sheet, a membrane, or a film.

Accordingly, in some aspects, disclosed herein is a method of creating an epithelial-endothelial barrier, said method comprising:
providing a substrate in an upright position, wherein said substrate has a first surface and a second surface, and wherein the second surface is located at an underside of the substrate when said substrate is in an upright position;
providing a first mixture of cells that comprises a plurality of epithelial cells and a cell suspension medium, wherein said cell suspension medium has a higher density than the epithelial cells;
contacting the first mixture of cells with the second surface, thereby seeding the underside of the substrate with the epithelial cells;
adding a second mixture of cells that comprises a plurality of endothelial cells and a cell culture medium on the first surface of the substrate;
removing the cell suspension medium to form a mixed cell culture having an air and liquid interface whereby the epithelial cells seeded on the underside of the substrate are exposed to the air, and whereby the endothelial cells on the first surface of the substrate are in contact with a cell culture medium, and
culturing the endothelial and epithelial cells at the air and liquid interface to form an epithelial-endothelial barrier, wherein the endothelial cells form an endothelial barrier on the first surface of the substrate, and the epithelial cells form an epithelial barrier on the underside of the substrate.

In some embodiments, the upright position of the substrate is maintained throughout the process of said method.

In some embodiments, the substrate is porous between the first surface and the second surface. In some embodiments, the pore size is about 0.1 μm, 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 9 μm, 10 μm, 15 μm, 20 μm, 40 μm, 60 μm, 80 μm, 100 μm, 150 μm, or 200 μm. In some embodiments, the pore size is about 0.1 μm-1 μm. In some embodiments, the pore size is about 0.5 μm to 5 μm. In some embodiments, the pore size is about 2 μm to 20 μm. In some embodiments, the pore size is about 10 μm to 100 μm. In some embodiments, the pore size is about 50 μm to 200 μm. In some embodiments, the pore size is about 50 μm to 500 μm.

In some embodiments, the first surface is located at an upperside of the substrate when said substrate is in an upright position. In some embodiments, the first and second surfaces of the substrate are parallel or non-parallel to one another. In some embodiments, at least one of the first surface and second surface is a textured surface. In some embodiments, at least one of the first surface and second surfaces is an angled surface. In some embodiments, at least one of the first surface and second surface is a curved surface. In some embodiments, the substrate is a sheet, a membrane, or a film.

Accordingly, in some examples, the substrate disclosed herein is a sheet, membrane, or a film, wherein the first surface is located at an upperside of the substrate when said substrate is in an upright position, wherein the second surface is located at an underside of the substrate when said substrate is in an upright position, and wherein the substrate is porous between the first surface and the second surface.

In some embodiments, the substrate is coated in whole or in part with an active agent (e.g., a biocompatible polymer, peptide, protein, or small molecule) that functions to promote cell adhesion or integration to the substrate. Biocompatible refers to materials that do not have toxic or injurious effects on biological functions. In some embodiments, the substrate is coated with collagen. In some embodiments, the substrate is coated with fibrinogen. In some embodiments, the substrate is coated with Matrigel.

In some embodiments, the cell suspension medium comprises one or more of:
a polymer or mixture of polymers;
a small molecule or mixture of small molecules;
a synthetic or naturally derived particle or mixture of particles;
a coacervate;
a colloid;
a protein or mixture thereof; and
a hydrogel or preparation thereof.

In some embodiments, the cell suspension medium further comprises a carrier solution selected from the group consisting of water, a buffer, a salt solution, and a cell culture medium.

In some embodiments, the cell suspension medium includes a dextran solution, a density gradient medium solution or a combination thereof. In some embodiments, the density gradient medium solution is an iodixanol solution, such as Optiprep®. The cell suspension medium is denser than the cells but can be tolerated by the contacted cells. Accordingly, in some embodiments, the cell suspension medium is denser than the cell by at least about 0.001 g/mL, 0.002 g/mL, 0.004 g/mL, 0.008 g/mL, 0.01 g/mL, 0.02 g/mL, 0.04 g/mL, 0.05 g/mL, 0.06 g/mL, 0.07 g/mL, 0.08 g/mL, 0.09 g/mL, 0.1 g/mL, 0.2 g/mL, 0.3 g/mL, 0.4 g/mL, 0.5 g/mL, 0.6 g/mL, 0.7 g/mL, 0.8 g/mL, 0.9 g/mL, 1.0 g/mL, 1.2 g/mL, 1.4 g/mL, 1.6 g/mL, 1.8 g/mL, or 2.0 g/mL. In some embodiments, the cells' density is about 1.05 g/mL. In some embodiments, the density of the cell suspension medium is about 1.06 g/mL, 1.07 g/mL, 1.08 g/mL, 1.09 g/mL, 1.10 g/mL, 1.11 g/mL, 1.12 g/mL, 1.13 g/mL, 1.14 g/mL, 1.15 g/mL, 1.16 g/mL, 1.17 g/mL, 1.18 g/mL, 1.19 g/mL, 1.2 g/mL, 1.22 g/mL, 1.24 g/mL, 1.26 g/mL, 1.28 g/mL, 1.3 g/mL, 1.35 g/mL, 1.4 g/mL, 1.45 g/mL, 1.5 g/mL, 1.55 g/mL, 1.6 g/mL, 1.65 g/mL, 1.7 g/mL, 1.8 g/mL, 1.9 g/mL, 2.0 g/mL, 2.2 g/mL, 2.3 g/mL, 2.4 g/mL, 2.5 g/mL, 2.6 g/mL, 2.7 g/mL, 2.8 g/mL, 2.9 g/mL, or 3.0 g/mL. Because of the difference in density, the cells (e.g., epithelial cells) can be floating on the surface of the cell suspension medium.

The first mixture of cells can be contacted with the second surface of the substrate for at least about 10, 20, 30, 40, or 50 min or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 hours. In some embodiments, the first mixture of cells is contacted with the second surface of the substrate for at least about 2 hours. In some examples, following the step of contacting the first mixture of cells with the second surface of the substrate, the cell suspension medium is diluted, and the seeded cells are incubated in the diluted cell culture medium for at least about 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 60 hours (“about” can refer to ±5 minutes, ±15 minutes or ±30 minutes).

Following the step of removing the cell suspension medium, the cells can be cultured at the air and liquid interface, whereby the adherent cells (e.g., epithelial cells) seeded on the underside of the substrate are exposed to the air, and whereby the cells on the first surface of the substrate (e.g., the endothelial cells) are in contact with a cell culture medium. In some examples, the cell culture medium in contact with the cells on the first surface (e.g., endothelial cells) is a serum-free, glucocorticoid-free medium. In some embodiments, the cell culture medium further comprises an Ultroser G serum substitute.

In some embodiments, the adherent cell (e.g., epithelial cells) on seeded on the underside surface of the substrate and the cells on the first surface of the substrate (endothelial cells) are cultured (e.g., cultured in liquid or at an air-liquid interface, or exposed to the air) for at least 3 days. In some embodiments, the adherent cell (e.g., epithelial cells) on seeded on the underside surface of the substrate and the cells on the first surface of the substrate (endothelial cells) are cultured at the air and liquid interface for at least 3 days (e.g., at least 3, 4, 5, 6, 7, 8, 9, 10, 11, 12, 13, 14, 15, 16, 17, 18, 19, or 20 days) to form an epithelial-endothelial barrier, wherein the endothelial cells form an endothelial barrier on the first surface of the substrate and the epithelial cells form an epithelial barrier on the underside of the substrate. In some embodiments, the seeded epithelial cells are exposed to the air for at least 3 days (e.g., at least 3, 4, 5, 6, 7, 8, 9, 10, 11, 12, 13, 14, 15, 16, 17, 18, 19, or 20 days). In some embodiments, the seeded epithelial cells are exposed to the air for at least 5 days. In some embodiments, the seeded epithelial cells are exposed to the air for at least 7 days.

In some embodiments, the seeded epithelial cells are differentiated by exposure to the air-liquid interface and exposure to differentiation compound Ultroser G (USG) that is present in the air-liquid interface culture medium formulation. In some embodiments, the endothelial cells are mature and confluent following culturing at the air-liquid interface. In some embodiments, the epithelial cells of the epithelial barrier have an increased level of a marker relative to a reference control, wherein the marker is selected from the group consisting of E-cadherin, ZO-1, e-cadherin, VE-cadherin, TMPRSS2, F-Actin, and a combination thereof.

In some embodiments, the epithelial-endothelial barrier has an improved barrier function relative to a reference control (e.g., a monoculture of epithelial cells or endothelial cells). The barrier function is determined by a measurement of trans-epithelial electrical resistance. In some embodiments, the epithelial-endothelial barrier disclosed herein is about at least about 20%, or at least about 30%, or at least about 40%, or at least about 50%, or at least about 60%, or at least about 70%, or at least about 80%, or at least about 90% or up to and including a 100% increase, or at least about a 2-fold, or at least about a 3-fold, or at least about a 4-fold, or at least about a 5-fold or at least about a 10-fold increase in the transepithelial electrical resistant as compared to a reference control (e.g., a monoculture of epithelial cells or endothelial cells). In some embodiments, the transepithelial electrical resistance of the epithelial-endothelial barrier is at least about 100 Ω·cm² (e.g., at least about 100 Ω·cm², 200 Ω·cm², 300 Ω·cm², 400 Ω·cm², 500 Ω·cm², 700 Ω·cm², 800 Ω·cm², 1000 Ω·cm², 1200 Ω·cm², 1400 Ω·cm², 1600 Ω·cm², 1800 Ω·cm², 2000 Ω·cm², 2200 Ω·cm², 2400 Ω·cm², 2600 Ω·cm², 2800 Ω·cm², or 3000 Ω·cm²). In some embodiments, the transepithelial electrical resistance of the epithelial-endothelial barrier is at least about 300 Ω·cm² on day 5, day 6, or day 7 from the start of culturing the endothelial and epithelial cells at the air and liquid interface.

In some embodiments, the surface is placed in or is a part of a multiple-well pate (e.g., a 6-well plate, a 12-well plate, a 24-well plate, a 48-well plate, a 96-well plate, or a 384-well plate). In some embodiments, the surface is placed in or is a part of a 96-well plate. In some embodiments, the surface is placed in or is a part of a 384-well plate. In some embodiments, the surface is placed in or is a part of a 384-pillar plate. In some embodiments, the second surface of the substrate has a surface area of about 0.02 cm², 0.05 cm², 0.1 cm², 0.15 cm², 0.2 cm², 0.3 cm², 0.4 cm², 0.5 cm², 0.8 cm², 1 cm², 1.5 cm², or 2 cm². In some embodiments, the substrate has a surface area of about 0.15 cm². In some embodiments, the substrate has a surface area of about 0.05 cm².

In some embodiments, the epithelial cell in the methods disclosed herein is a cell line, an engineered epithelial cell, or a primary epithelial cell. In some embodiments, the epithelial cell is an H441 club cell line. In some embodiments, the epithelial cell is a cell in a hollow organ (e.g., stomach, intestine, gallbladder, or bladder). In some embodiments, the epithelial cell is a lung epithelial cell.

Components, systems, and methods can be used with both animal cells and human cells, and non-animal cells such as insect or plant cells, and methods may comprise cross-species extrapolation. For example, endothelial/epithelial cells can be used in the assay. Also disclosed are one or more of the following cell types, either alone or in combination: cardiac myocytes, a hepatic component comprising liver cells, a gastrointestinal component comprising epithelial cells and/or mucus-producing cells, a muscular component comprising muscle cells, a kidney-like filtering component, a neural component, a neuromuscular component and/or other components analogous to body structures, organs or organ systems, and optionally, further comprising a housing for enclosing the components or a board for immobilizing components.

### An In Vitro Tool, System, Apparatus, and Uses Thereof for Screening for Active Agents

In some aspects, disclosed herein is an in vitro tool, system, and/or apparatus for screening or evaluating active agents that modulate a cell barrier or that modulate cells crossing the aforementioned barrier and/or diagnosing a disease, said in vitro tool, system, and/or apparatus is prepared by the method disclosed herein. In some embodiments, the adherent cells are epithelial cells or fibroblasts.

In some embodiments, the method comprises:
providing a substrate in an upright position, wherein said substrate has a first surface and a second surface, wherein the second surface is located at an underside of the substrate when said substrate is in an upright position;
providing a first mixture of cells that comprises a plurality of adherent cells (e.g., epithelial cells) and a cell suspension medium, wherein said cell suspension medium has a higher density than the adherent cells (e.g., epithelial cells);
contacting the first mixture of cells with the second surface, thereby seeding the underside of the substrate with the adherent cells (e.g., epithelial cells);
adding a second mixture of cells that comprises a plurality of cells (e.g., endothelial cells) and a cell culture medium on the first surface of the substrate;
removing the cell suspension medium to form a mixed cell culture having an air and liquid interface whereby the adherent cells (e.g., epithelial cells) seeded on the underside of the substrate are exposed to the air, and whereby the cells on the first surface of the substrate (e.g., endothelial cells) are in contact with a cell culture medium, and
culturing the adherent cell (e.g., epithelial cells) on seeded on the underside surface of the substrate and the cells on the first surface of the substrate (endothelial cells).

In some embodiments, the method comprises:
providing a substrate in an upright position, wherein said substrate has a first surface and a second surface, wherein the second surface is located at an underside of the substrate when said substrate is in an upright position;
providing a first mixture of cells that comprises a plurality of epithelial cells and a cell suspension medium, wherein said cell suspension medium has a higher density than the epithelial cells;
contacting the first mixture of cells with the second surface, thereby seeding the underside of the substrate with the epithelial cells;
adding a second mixture of cells that comprises a plurality of endothelial cells and a cell culture medium on the first surface of the substrate;
removing the cell suspension medium to form a mixed cell culture having an air and liquid interface whereby the epithelial cells seeded on the underside of the substrate are exposed to the air, and whereby the endothelial cells on the first surface of the substrate are in contact with a cell culture medium, and
culturing the endothelial and epithelial cells at the air and liquid interface to form an epithelial-endothelial barrier, wherein the endothelial cells form an endothelial barrier on the first surface of the substrate, and the epithelial cells form an epithelial barrier on the underside of the substrate.

In some embodiments, the in vitro tool, system, and/or apparatus comprises
a porous substrate configurable to an upright position,
an endothelial barrier, and
an epithelial barrier,
wherein the endothelial barrier is formed on a first surface of the substrate, and the epithelial barrier is formed on a second surface of the substrate, wherein the second surface is located at an underside of the substrate.

In some embodiments, the substrate is porous between the first surface and the second surface. In some embodiments, the pore size is about 0.1 μm, 0.5 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 9 μm, 10 μm, 15 μm, 20 μm, 40 μm, 60 μm, 80 μm, 100 μm, 150 μm, or 200 μm. In some embodiments, the pore size is about 0.1 μm-1 μm. In some embodiments, the pore size is about 0.5 μm to 5 μm. In some embodiments, the pore size is about 2 μm to 20 μm. In some embodiments, the pore size is about 10 μm to 100 μm. In some embodiments, the pore size is about 50 μm to 200 μm. In some embodiments, the pore size is about 50 μm to 500 μm. In some embodiments, the pore size is about 3 μm.

In some embodiments, the first surface is located at an upperside of the substrate when said substrate is in an upright position. In some embodiments, the first and second surfaces of the substrate are parallel or non-parallel to one another. In some embodiments, at least one of the first surface and second surface is a textured surface. In some embodiments, at least one of the first surface and second surfaces is an angled surface. In some embodiments, at least one of the first surface and second surface is a curved surface. In some embodiments, the substrate is a sheet, a membrane, or a film.

In some examples, the substrate disclosed herein is a sheet, membrane, or a film, wherein the first surface is located at an upperside of the substrate when said substrate is in an upright position, wherein the second surface is located at an underside of the substrate when said substrate is in an upright position, and wherein the substrate is porous between the first surface and the second surface.

In some embodiments, the in vitro tool comprises a porous membrane, film, or sheet comprising an endothelial barrier and an epithelial barrier on the opposite sides of the membrane, film, or sheet.

In some embodiments, the substrate is coated in whole or in part with an active agent (e.g., a biocompatible polymer, peptide, protein, or small molecule) that functions to promote cell adhesion or integration to the substrate. Biocompatible refers to materials that do not have toxic or injurious effects on biological functions. In some embodiments, the substrate is coated with collagen. In some embodiments, the substrate is coated with fibrinogen. In some embodiments, the substrate is coated with Matrigel.

In some embodiments, the transepithelial electrical resistance of the in vitro tool (e.g., a membrane, film, or sheet comprising an endothelial barrier and an epithelial barrier on the opposite sides of the membrane, film, or sheet) is at least about 100 Ω·cm² (e.g., at least about 100 Ω·cm², 200 Ω·cm², 300 Ω·cm², 400 Ω·cm², 500 Ω·cm², 700 Ω·cm², 800 Ω·cm², 1000 Ω·cm², 1200 Ω·cm², 1400 Ω·cm², 1600 Ω·cm², 1800 Ω·cm², 2000 Ω·cm², 2200 Ω·cm², 2400 Ω·cm², 2600 Ω·cm², 2800 Ω·cm², or 3000 Ω·cm²). In some embodiments, the transepithelial electrical resistance of the in vitro tool is at least about 300 Ω·cm². In some embodiments, the transepithelial electrical resistance of the in vitro tool is at least about 500 Ω·cm²

In some embodiments, the in vitro tool comprising the substrate disclosed herein is placed in or is a part of a multiple-well pate (e.g., a 6-well plate, a 12-well plate, a 24-well plate, a 48-well plate, a 96-well plate, or a 384-well plate or other plates disclosed herein). In some embodiments, the surface is placed in or is a part of a 96-well plate. In some embodiments, the in vitro tool comprising the epithelial-endothelial barrier is placed in or is a part of a 384-well plate or a 384 pillar plate. In some embodiments, the second surface of the substrate of the substrate has a surface area of about 0.02 cm², 0.05 cm², 0.1 cm², 0.15 cm², 0.2 cm², 0.3 cm², 0.4 cm², 0.5 cm², 0.8 cm², 1 cm², 1.5 cm², or 2 cm². In some embodiments, the second surface of the substrate has a surface area of 0.143 cm². In some embodiments, the second surface of the substrate has a surface area of 0.05 cm². In some embodiments, the distance between the underside surface of the substrate and the bottom of the well is from about 0.2 mm to about 5 mm. In some embodiments, the distance between the underside surface of the substrate and the bottom of the well is less than 2 mm (e.g., less than 1.5 mm, less than 1.3 mm, less than 1 mm, less than 0.8 mm, less than 0.5 mm, or less than 0.2 mm). In some embodiments, the distance between the underside surface of the substrate and the bottom of the well is less than 1.3 mm. In some embodiments, the distance between the underside surface of the substrate and the bottom of the well is about 0.5 mm.

The assay and method disclosed herein can be used to measure the effect of an input variable on the cells in the culture. Examples of input variables include, but are not limited to, test compounds, molecules, reagents, or organisms. The input variable can be an organic or inorganic chemical compound. An input variable may be more than one compound and may be a mixture of inorganic and organic compounds. An input variable may be a pharmaceutical composition, an environmental sample, a nutritional sample, or a consumer product. An input variable may be a virus, liposome, nanoparticle, biodegradable polymer, radiolabeled particle or toxin, biomolecule, toxin-conjugated particle or biomolecule, or a combination thereof. The time period for testing the reaction of one or a plurality of components in a cell culture analog system may be for 72 hours, 84 hours, 96 hours, 108 hours, 120 hours, 132 hours, 144 hours, 156 hours, 168 hours, 180 hours, or for days or weeks, or longer, or any amount of time in between.

Any cell culture plate known to those of skill in the art can be used with the methods and assays disclosed herein. Some embodiments of the invention may use Transwell™ plates from Corning. In one configuration, the plates may have wells (e.g., 24 wells among other disclosed herein) arranged in a rectangular array of the same footprint as a standard microtiter plate. Each plate may include i) a bottom part with multi-cylindrical wells (e.g., 24, etc.); ii) a middle part consisting of multiple Transwells™ (e.g., 24, etc.), each of which is a cup whose bottom is a microporous membrane support on which epithelial cells can grow; and iii) a lid. The middle part also has access holes adjacent to each Transwell™ which pass through the tray to allow pipetting into and out of the bottom wells. The microporous membrane support may be made of PTFE, polyester, or polycarbonate and, e.g., has pore sizes ranging from 0.1 to 8 μm; the area can be 0.33 cm² or other sizes as described herein. When referring to the “underside,” the term refers to the underside of the bottom plate.

Also disclosed herein is a method of screening for or evaluating active agents that modulate a cell barrier or modulate cells capable of crossing said barrier or diagnosing a disease, said method comprising:
providing the in vitro tool, system, or apparatus disclosed herein;
contacting the in vitro tool with an active agent;
contacting the barrier on the first surface of the in vitro tool with a plurality of cells (e.g., immune cells or non-immune cells), wherein the barrier comprises adherent cells,
contacting the barrier on the second surface of the in vitro tool with a fluid sample, wherein the barrier comprises adherent cells; and
measuring the number of the cells transmigrating to the side of the second surface and/or determining a barrier function;
wherein an indication that the active agent impairs or improves the barrier includes at least one of a change in the number of the cells transmigrating to the side of the barrier on the second surface of the substrate or a change in the barrier function. In some embodiments, the barrier on the first surface is an endothelial barrier. In some embodiments, the barrier on the second surface is an epithelial barrier.

In some embodiments, the in vitro tool, system, or apparatus is prepared by the method disclosed herein.

Accordingly, disclosed herein is a method of screening for or evaluating active agents that modulate a barrier (e.g., an epithelial barrier or an epithelial-endothelial barrier) or diagnosing a disease, said method comprising:
providing a substrate in an upright position, wherein said substrate has a first surface and a second surface, wherein the second surface is located at an underside of the substrate when said substrate is in an upright position;
providing a first mixture of cells that comprises a plurality of adherent cells (e.g., epithelial cells) and a cell suspension medium, wherein said cell suspension medium has a higher density than the adherent cells (e.g., epithelial cells);
contacting the first mixture of cells with the second surface, thereby seeding the underside of the substrate with the adherent cells (e.g., epithelial cells);
adding a second mixture of cells that comprises a plurality of cells and a cell culture medium on the first surface of the substrate;
removing the cell suspension medium to form a mixed cell culture having an air and liquid interface whereby the adherent cells (e.g., epithelial cells) seeded on the underside of the substrate are exposed to the air, and whereby the cells on the first surface of the substrate are in contact with a cell culture medium;
culturing the cells on the first surface and cells on the second surface at the air and liquid interface to form a cell barrier;
contacting the cell barrier with an active agent;
contacting the barrier on the first surface with a plurality of cells;
contacting the barrier on the second surface with a fluid sample; and measuring the number of the cells transmigrating to the side of the barrier on the second surface and/or determining a barrier function;
   wherein an indication that the active agent impairs or improves the epithelial barrier includes at least one of a change in the number of the cells transmigrating to the side of the epithelial barrier or a change in the barrier function or barrier characteristics including expression of proteins, small molecules, production of chemical compounds or other biological material.

Also disclosed herein is a method of screening for active agents that modulate a cell barrier, evaluating therapeutic effects of active agents, or diagnosing a disease, said method comprising:
providing a substrate in an upright position, wherein said substrate having a first surface and a second surface, wherein the second surface is located at an underside of the substrate when said substrate is in an upright position;
providing a first mixture of cells that comprises a plurality of epithelial cells and a cell suspension medium, wherein said cell suspension medium has a higher density than the epithelial cells; and
contacting the first mixture of cells with the second surface, thereby seeding the underside of the substrate with the epithelial cells;
adding a second mixture of cells that comprises a plurality of endothelial cells and a cell culture medium on first surface of the substrate;
removing the cell suspension medium to form a mixed cell culture having an air and liquid interface whereby the epithelial cells seeded on the underside of the substrate are exposed to the air and endothelial cells on the first surface of the substrate are in contact with a cell culture medium,
culturing the endothelial and epithelial cells at the air and liquid interface to form an epithelial-endothelial barrier, wherein the endothelial cells form an endothelial barrier on the first surface of the substrate and the epithelial cells form an epithelial barrier on the underside of the substrate,
contacting the epithelial-endothelial barrier with an active agent;
contacting the endothelial barrier of the epithelial-endothelial barrier with a plurality of cells,
contacting the epithelial barrier with a fluid sample; and
measuring the number of the cells transmigrating to the side of the epithelial barrier and/or determining a barrier function;
   wherein an indication that the active agent impairs or improves the epithelial barrier includes at least one of a change in the number or activation state of the cells transmigrating to the side of the epithelial barrier or a change in the barrier function or barrier characteristics including expression of proteins, small molecules, production of chemical compounds or other biological material.

In some embodiments, an increase in the number of the cells transmigrating to the side of the barrier on the second surface or a decrease in the barrier function is an indication that the active agent is impairing the cell barrier.

In some embodiments, a decrease in the number of the cells transmigrating to the side of the barrier on the second surface or an increase in the barrier function is an indication that the active agent improves the cell barrier. In some embodiments, a decrease in the number of the cells transmigrating to the side of the epithelial barrier or an increase in the barrier function is an indication that the active agent improves the epithelial barrier. In some embodiments, a decrease in the number of the cells transmigrating to the side of the epithelial barrier or an increase in the barrier function is an indication that the active agent improves the distal lung environment.

In some embodiments, the cells are immune cells or non-immune cells. In some embodiments, the immune cells are isolated from peripheral blood (e.g., white blood cells). In some embodiments, the immune cells are neutrophils. In some embodiments, the non-immune cells are cancer cells.

In some embodiments, the fluid sample comprises a chemoattractant (e.g., including IL-8 or LTB4) or a pro-inflammatory substance (e.g., including neutrophil extracellular trap (NET)-mimic chromatin). In some embodiments, the chemoattractant comprises TNF-α, IL-1α, IL-1β, lipopolylsaccharide, poly(I:C), bleomycin, soluble organic aerosols, CCL2, MIP-1α, C5a, C3a, C3b, or extracellular DNA.

In some embodiments, the fluid sample comprises biological fluids obtained from a healthy subject or a patient having a lung disorder (e.g., chronic obstructive pulmonary disease, acute respiratory distress syndrome (ARDS), cystic fibrosis (CF), or a combination thereof).

The active agent can be introduced to the barrier on the second surface and/or the barrier on the first surface of the in vitro tool. In some embodiments, the active agent can be introduced to the barrier on the second surface and the barrier on the first surface. In some embodiments, the active agent can be introduced to the barrier on the second surface. In some embodiments, the active agent can be introduced to the barrier on the first surface. The active agent can be introduced to the barrier on the second and/or the first surface via an aerosol (e.g., through exposing the barrier on the second and/or the first surface to the air comprising the active agent) or a fluid (e.g., through contacting the barrier on the second and/or the first surface to the fluid comprising the active agent).

In some embodiments, the active agent includes a chemical compound, a molecule, a toxin, or an organism. In some embodiments, the toxin is a substance of a tobacco product. In some embodiments, the toxin is a pollution particle. In some embodiments, the active agent is a therapeutic agent. In some embodiments, the organism is a pathogen (e.g., a virus such as influenza virus or a coronavirus).

Accordingly, in some aspects, disclosed herein is a method of screening for or evaluating toxins or organisms that modulate a cell barrier or modulate cells capable of migrating through said barrier, said method comprising:
providing the in vitro tool, system, or apparatus disclosed herein or an in vitro tool, system, or apparatus prepared by the methods disclosed herein;
contacting the in vitro tool, system, or apparatus with a toxin or organism;
contacting the barrier on the first surface of the in vitro tool, system, or apparatus with a plurality of cells,
contacting the barrier on the second surface of the in vitro tool, system, or apparatus with a fluid sample; and
measuring the number of the cells transmigrating to the side of the barrier on the second surface and/or determining a barrier function;
   wherein an increase in the number of the cells transmigrating to the side of the barrier on the second surface or a decrease in the barrier function is an indication that the toxin or organism impairs the barrier; and
   wherein a decrease in the number of the cells transmigrating to the side of the barrier on the second surface or an increase in the barrier function is an indication that the toxin or organism improves the barrier.

In some embodiments, the organism is a pathogen. In some the pathogen is a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2. In some embodiments, the pathogen is an influenza virus or SARS-CoV-2.

In some aspects, disclosed herein is a method of screening for or evaluating therapeutic agents that modulate a cell barrier or modulate cells capable of migrating throught said barrier, said method comprising:
providing the in vitro tool, system, or apparatus disclosed herein or an in vitro tool, system, or apparatus prepared by the methods disclosed herein;
contacting the in vitro tool, system, or apparatus with a therapeutic agent;
contacting the barrier on the first surface of the in vitro tool, system, or apparatus with a plurality of cells;
contacting the barrier on the second surface of the in vitro tool, system, or apparatus with a fluid sample; and
measuring the number of the cells transmigrating to the side of the barrier on the second surface and/or determining a barrier function;
   wherein an increase in the number of the cells transmigrating to the side of the barrier on the second surface or a decrease in the barrier function is an indication that the therapeutic agent impairs the barrier; and
   wherein a decrease in the number of the cells transmigrating to the side of the barrier on the second surface or an increase in the barrier function is an indication that the therapeutic agent improves the barrier.

Also, in some embodiments, the cells (e.g., immune cells) added to the endothelial barrier of the in vitro tool can be pre-treated with a therapeutic agent.

Accordingly, in some aspects, disclosed herein is a method of screening for or evaluating therapeutic agents that modulate a cell barrier or modulate cells capable of migrating through said barrier, said method comprising:
providing the in vitro tool, system, or apparatus disclosed herein or an in vitro tool, system, or apparatus prepared by the methods disclosed herein;
contacting the barrier on the first surface of the in vitro tool, system, or apparatus with a mixture comprising a plurality of cells and a therapeutic agent;
contacting the barrier on the second surface with a fluid sample; and
measuring the number of the cells transmigrating to the side of the barrier on the second surface and/or determining a barrier function;
   wherein a decrease in the number of the cells transmigrating to the side of the barrier on the second surface or an increase in the barrier function is an indication that the therapeutic agent improves the barrier.

In some embodiments, the cells are immune cells. In some embodiments, the immune cells are neutrophils.

In some embodiments, the method further comprises determining levels of one or more immune cell activation markers on the transmigrating cells, wherein a decrease in the levels of the one or more markers indicates that the active agent deactivates immune cells, or wherein an increase in the levels of the one or more markers indicates that active agent activates immune cells.

Also, in some aspects, disclosed herein is a method of diagnosing and/or studying a lung associated disorder in a subject, said method comprising
obtaining a fluid sample from the subject,
providing the in vitro tool, system, or apparatus disclosed herein or an in vitro tool, system, or apparatus prepared by the methods disclosed herein;
contacting the endothelial barrier of the in vitro tool, system, or apparatus with a plurality of immune cells (e.g., neutrophils),
contacting the epithelial barrier with the fluid sample;
measuring the number of the immune cells transmigrating to the side of the epithelial barrier and determining the barrier function;
   wherein an increase in the number of immune cells transmigrating to the side of the epithelial barrier and/or a decrease in the barrier function is an indication that the subject has a lung associated disorder.

As described above, in some embodiments, the substrate comprising the epithelial-endothelial barrier can be placed in or a part of a multiple-well plate (e.g., a 96-well plate, a 384-well plate, a Transwell 96 plate, or a 384-pillar plate or other plates described herein). Accordingly, less volume (e.g., as little as 80 microliter) of a patient fluid sample may be employed in the lower epithelial side chamber. This volume is typically on the order of milliliters. Thus, the method disclosed herein facilitate the study of a single patient at a time while other studies may mix many patient samples into one experiment well. In some embodiments, the volume of the fluid sample is about at least about 10 microliter, 20 microliter, 40 microliter, 80 microliter, 100 microliter, or 200 microliter. In some embodiments, the fluid sample is less than 100 microliter (e.g., about 10 microliter, 20 microliter, 40 microliter, or 80 microliter)

In some embodiments, the fluid sample comprises biological fluids obtained from a healthy subject or a patient having a lung disorder (e.g., chronic obstructive pulmonary disease, acute respiratory distress syndrome (ARDS), cystic fibrosis (CF), or a combination thereof).

In some embodiments, the evaluated disease is a lung associated disorder such as chronic obstructive pulmonary disease, acute respiratory distress syndrome (ARDS), cystic fibrosis (CF), community-acquired pneumonia, acute hypersensitivity pneumonitis, asthma, acute or chronic eosinophilic pneumonia, respiratory bronchiolitis, a coronavirus-associated lung disorder (e.g., COVID-19), an influenza virus-associated lung disorder, or a combination thereof.

In some embodiments, the method further comprises administering a therapeutically effective amount of a therapeutic agent to the subject who is diagnosed as having a lung associated disorder.

In some embodiments, the method further comprises determining levels of one or more markers on a transmigrating cell (e.g., transmigrating neutrophils). In some embodiments, the one or more markers are selected from the group consisting of CD62L, CD66b, CD16, CD63, CD49d, HLA-DR, CD181, CD86, CD182, CD54, CD56, CD14, CD3, CD15, CD45, HNE, CD184, CD41a, CD11b, Arg1, CD11c, and PD-L1. In some embodiments, one or more markers are selected from the group consisting of CD62L, CD66b, CD16, and CD63.

### EXAMPLES

### Example 1. Method of Attachment to the Underside of a Surface by Exploitation of Density to Drive Buoyancy

Background High-throughput cell culture models enable the rapid testing of large condition sets for the accelerated study of cellular response to pathogens, toxins, and therapeutics. However, tissue barrier models typically use 2D monocultures that incompletely capture the physiologic conditions that produce complex in vivo responses to stimuli. In particular, the lung's air-blood barrier is comprised of epithelial cell plus endothelial cell layers that together form a critical first line of defense against infections and toxins and act as a regulator of access to the bloodstream. Despite this, the lung barrier is frequently modeled in high-throughput (96- or more wells per plate) with only the epithelium. While epithelial tight junctions can provide strong permeability restriction in the lung, the underlying endothelium can potentiate or compromise epithelial barrier function, e.g., by paracrine signaling. In fact, acute lung injury characterized by loss of epithelial barrier function can originate from endothelial activation and dysfunction rather than direct epithelial injury.

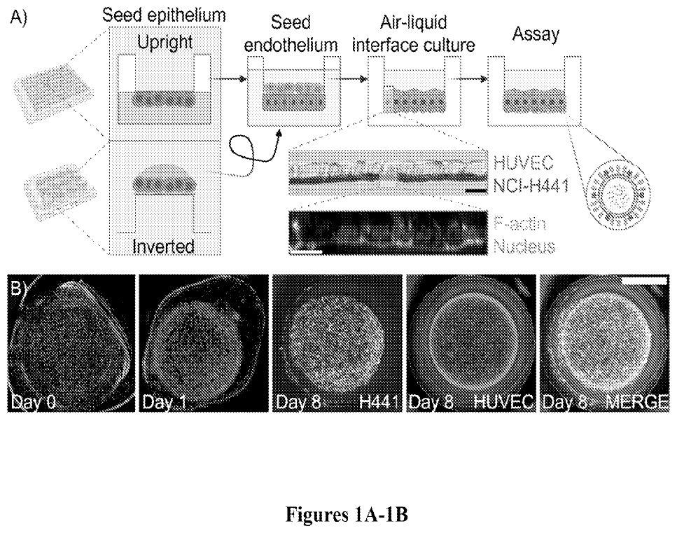

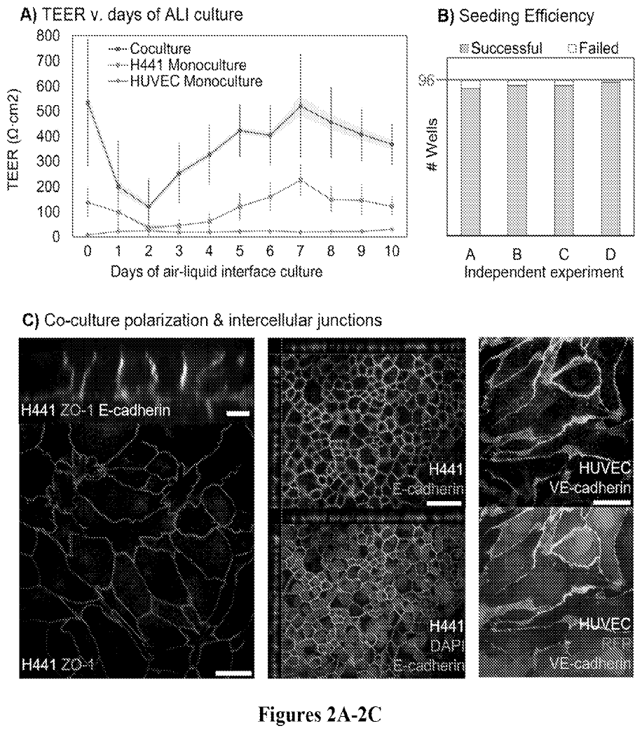

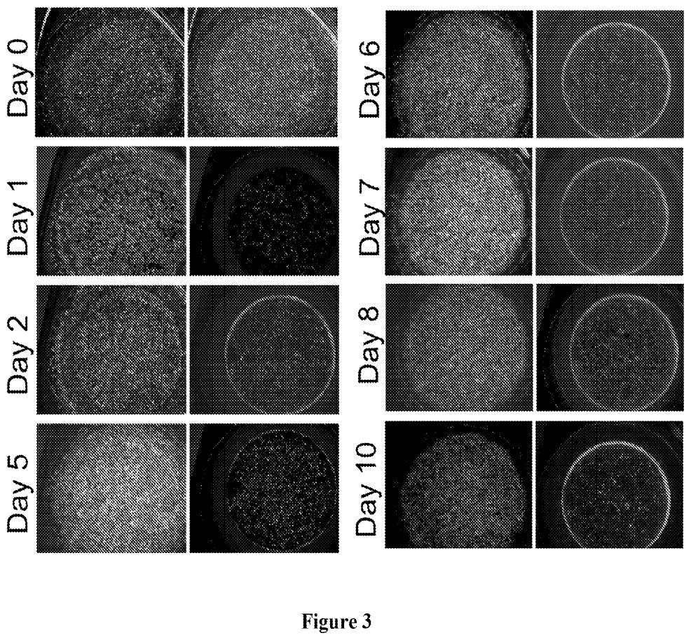

Therefore, lung air-blood barrier models based on an epithelial cell monoculture are not optimal models to emulate barrier function due to the absence of a co-cultured endothelium. Co-culture, however, requires the culture of two cell types on opposite sides of a culture membrane. While it is possible to culture endothelium in the underlying well rather than opposite the epithelium, there is evidence that the barrier function of the epithelium is a combination of cell-cell contact and paracrine signaling so that co-culture on opposite sides of a membrane is required to fully reproduce the barrier function of co-culture. Such co-culture is typically performed in 6- to 24-well plates where the underside of the culture inserts is seeded by manual inversion of the inserts (FIG. 1). Such inversion procedures can be tedious, difficult to automate, and prone to failure, particularly for large number of well inserts (e.g., 96 well inserts) due to the limited culture area. Specifically, difficulties may arise from the limited volume of liquid (˜30 μL) that can be placed on inverted 96-well membrane inserts, meaning that cells must be very concentrated, and the risks of evaporation and contamination during cell attachment are not insignificant.

To address these challenges, disclosed herein is a method that eliminates the need for inversion by exploiting density-driven cell floating for underside cell seeding. This technique can then be used to develop a robust tissue barrier platform with scalable, automatable co-culture that requires no plate inversion and uses commercially available reagents and liquid handling equipment. In a model of the small airway epithelial-endothelial barrier, co-culture may be necessary for complete barrier strength, and communication between cell types in the co-culture can mediate the response to inflammatory pathogens. The model can recapitulate tissue features, including low permeability, high trans-epithelial electrical resistance (TEER), and epithelial-endothelial communication and loss of barrier function, e.g., in response to inflammatory stimuli. The capability of epithelial culture was demonstrated on the underside of the membrane under air-liquid interface culture (ALI) conditions with serum-free, glucocorticoid-free media. The exemplary seeding method is translatable to many vascular-epithelial tissue barriers and can be used to eliminate a bottleneck step of the bilayer co-culture process.

### Example Experimental Protocols

96-well Transwell Upright Seeding and Maintenance. Polycarbonate 96-well HTS Transwell Permeable Supports with pore size 3 μM were employed from Corning (CLS3386, CLS3382). The inserts were collagen-coated prior to cell seeding to promote attachment. Rat tail collagen type I (Corning 354236) was suspended at 30 μg/mL in 60 vol % ethanol (Fisher BP8203-1GAL) that was adjusted to pH 6 with hydrochloric acid (0.1 M) and diluted to 60% v/v in sterile distilled water. Inserts were inverted in a sterile biosafety cabinet, and 30.3 μL collagen solution was added to the underside of each insert with the VIAFLO-96 liquid handler (INTEGRA Biosciences #6001 and #6106). The inserts were allowed to dry overnight in the sterile biosafety cabinet.

Upright underside seeding. H441 cells were passaged and suspended at 1.18e6 cells/mL in warm cell culture media. The cell solution was gently mixed with sterile, pre-warmed (37° C.) 50 vol % OptiPrep™ Density gradient medium (STEMCELL Technologies 07820) until a homogeneous solution was observed. The solution was immediately transferred to the lower chamber of the HTS 96-well plates (85 μL/well) using a multichannel pipettor or VIAFLO-96 liquid handler, ensuring adequate mixing for even cell distribution. This concentration and volume results in 50,000 cells/well (350,000 cells/cm²). The cells adhered in this condition for 2 hours in a humid incubator at 37° C., 5% CO2, 95% humidity. After 2 hours, 150 μL cell culture media was slowly added to the bottom chamber, and 75 μL was added to the top chamber using a VIAFLO-96 Liquid Handler. The plate was then allowed to incubate overnight before HUVEC seeding.

HUVEC seeding. The day following H441 seeding, HUVECs were seeded in the Transwell chamber on the opposite side of the H441 cells. HUVEC-RFP cells were passaged and suspended at 80,000 cells/mL. A culture comprising 10,000 cells/well in 100 μL cell culture media was seeded in each well and allowed to incubate overnight.

ALI culture. Inserts were transitioned to ALI after >90% of wells were confluent upon manual inspection with epifluorescence microscopy. Typically this occurs 24-48 hours after HUVEC seeding. To culture at ALI, the media was removed from the bottom chamber, and the media in the top chamber was replaced with 50/50 vol % H441 and HUVEC cell culture media with all supplements except FBS. Instead of FBS, the media contained 1:50 v/v Ultroser G serum substitute (final concentration 0.2 mg/mL) (Sartorius 15950-017) to promote differentiation and polarization of the epithelium. The plate was then cultured at ALI for 5-7 days until the trans-epithelial electrical resistance reached above an average of 400 Ω·cm². Assays were typically performed on day 6 of ALI culture, day 9 since seeding H441.

Live Virus Exposure. To mimic viral infection of the epithelium, the study conducted a 24-hour exposure to synthetic viral RNA mimic Poly(I:C) on the epithelial side, the endothelium released inflammatory chemoattractant IL-8 that recruit immune cells from the vasculature in many respiratory pathologies in a weakly dose-dependent manner. This result shows that the assay is sensitive to signaling from the epithelial to endothelial side.

Further, the study reproduced the anti-inflammatory effect of the glucocorticoid dexamethasone, showing that IL-8 production by the endothelium is reduced after endothelial pre-treatment with dexamethasone on the endothelium. This assay was possible because the study differentiated the cell layers using an Ultroser G serum substitute that does not contain dexamethasone. Typical differentiation of the airway epithelium can be accomplished with dexamethasone, but the study transitioned to a serum-free, dexamethasone-free method to provide glucocorticoid assays such as this one and to eliminate the influence of low-dose glucocorticoid used for differentiation on the inflammatory signaling responses induced by exposures.

H441 maintenance in T-75 flasks. NCI-H441 human adenocarcinoma cell line was employed from American Type Culture Collection (ATCC) (ATCC® HTB-174™). H441 cells were expanded in RPMI-1640 (ATCC® 30-2001™) supplemented with 9% fetal bovine serum (50 mL into 500 mL media for total volume 550 mL), Penicillin-Streptomycin (Gibco™ 15140148) diluted 1:100 v:v, and 1.5 μg/mL puromycin. H441 cells were transduced to express GFP as described elsewhere in the Methods section. For routine culture, GFP-H441 cells were seeded at density 1e6 cells/75 cm² in 20 mL cell culture media. After passage (day 0), the media was changed every 48 hours on the following schedule: day 2, 20 mL; day 4, 40 mL; day 6, passage. For media changes, H441 media was aspirated, cells were rinsed with 10 mL warm phosphate-buffered saline (PBS) (Gibco™ 10010023), and media was replaced. For passage, H441 T-75s were rinsed with 10 mL warm PBS and lifted with 2 mL 0.05% Trypsin-EDTA (Gibco™ 25300120). Trypsin was neutralized with media, and cells were spun down (200 g, 5 min, 25° C.), resuspended in 1 mL media, and counted for seeding with Nexcelcom Cellometer Auto T4 Bright Field Cell Counter (Nexcelcom Bioscience) using Trypan Blue viability stain. Cells were used below the 8th passage after being obtained from ATCC.

HUVEC maintenance in T-75 flasks. Primary Human Umbilical Vein Endothelial Cells were employed from ATCC (ATCC® PCS-100-013™) and expanded according to manufacturer instructions in Vascular Cell Basal Medium (ATCC® PCS-100-030™) supplemented with Endothelial Cell Growth Kit-VEGF (ATCC® PCS-100-041™) with added Penicillin-Streptomycin (Gibco™ 15140148) diluted 1:100 v:v. HUVECs were transduced to constitutively express RFP. However, puromycin was not included in the routine cell culture medium because the cells did not tolerate it well for long time periods (unpublished observation). Cells were passaged at 60-80% confluence according to manufacturer instructions, counted with Nexcelcom Cellometer Auto T4 Bright Field Cell Counter (Nexcelcom Bioscience) using Trypan Blue viability stain, and used below the passage 10 since expanding from ATCC.

Lentiviral transduction. H441 cells and HUVECs were transduced with lentivirus to constitutively express GFP and RFP, respectively. H441 cells were seeded in T-75 flasks at 0.75e6 cells/75 cm² and allowed to attach overnight. Then H441 cells were inoculated with GFP gene-bearing lentivirus with puromycin resistance (Brand, product #). Lentivirus (8 TU/cell) was suspended in H441 routine culture media with the addition of transfection reagent polybrene (1 g/mL). The cells were inoculated overnight and then allowed to recover in routine cell culture media for 72 hours with media changes every 48 hours. Then, expressing cells were selected with puromycin: cells were cultured in routine culture media plus puromycin (1.5 μg/mL) until P3. H441-GFP cells were then frozen in puromycin-containing media with 0.05% cell culture-grade DMSO (ThermoFisher D12345). For RFP transduction of HUVECs, HUVECs were seeded at 8000 cells/cm cm² in 6-well plates and allowed to attach overnight. Then they were inoculated with 8 TU/cell RFP-gene bearing lentivirus in cell culture media supplemented with polybrene (1 g/mL) and incubated overnight. The viral media was removed, and the cells were allowed to grow in routine culture media for 72 hours with media changed every 48 hours. Then, RFP-expressing cells were selected by culturing in cell culture media supplemented with 1.5 μg/mL puromycin for 2 days. The selected cells were then expanded to P5 and frozen in puromycin-containing media with 0.05% cell culture-grade DMSO (ThermoFisher D12345). For a routine culture of HUVEC-RFP after thaw, puromycin was not included because HUVECs did not tolerate it for long time periods (unpublished observation).

Trans-epithelial electrical resistance (TEER). TEER was monitored daily beginning 48 hours after H441 seeding using EVOM2 0-10 kΩ Range Epithelial Volt/Ohm Meter (World Precision Instruments) with the STX100C96 electrode (World Precision Instruments). The electrode was maintained as recommended by the manufacturer. The electrode was cleaned by incubating overnight with Tergazyme (Alconox 1304-1), sanded gently as needed with sandpaper provided by the manufacturer to keep the metal surfaces clean and exposed, and was soaked as needed in 5% sodium hypochlorite for 5 minutes to maintain conductivity. Raw measurements of TEER were corrected according to the following formula: TEER (Ω·cm²)=[(Raw TEER value)−200 Ω]*0.143 cm². 200Ω is the average value of a blank well with the equivalent volume of cell culture media, while 0.143 cm² is the surface area of the cell culture insert. For TEER measurements of wells at ALI, 200 μL pre-warmed 37° C. PBS was added to the bottom chamber for measurement. For all TEER measurements, plates were placed on aluminum warming blocks to maintain constant temperature because TEER can change with temperature.

Staining and imaging. All stains used reagents from ThermoFisher's Image-iT™ Fixation/Permeabilization Kit (R37602) for washing, fixing, blocking, and permeabilization. For all stains, inserts were cut out and fixed on glass slides for 10 minutes at 37° C. For stains requiring permeabilization, the inserts were incubated with 0.5% Triton X (ImageIT kit) for 5 minutes at 37° C. All inserts were blocked for 1 hour at 37° C., counterstained with DAPI (1:1000 in PBS for 5 minutes) and mounted between two coverslips with ProLong™ Diamond Antifade Mountant (Invitrogen). F-actin. After fixation, permeabilization, and blocking, inserts were incubated for 1 hour at 37° C. in Alexa Fluor™ 647 Phalloidin (Invitrogen) diluted 1:20 from stock solution in 1% BSA (Image-iT™). ZO-1/e-cadherin co-stain. After fixation, permeabilization, and blocking, the primary antibodies (ZO-1 Rabbit anti-human polyclonal antibody, ThermoFisher 61-7300, 1:200; e-cadherin Mouse anti-human monoclonal antibody, ThermoFisher 13-1700, 1:2000) were suspended together in 1% BSA (Image-It Kit) and co-incubated with the inserts for 2 hours at 37° C. The secondary antibodies (Goat anti-Rabbit IgG, Alexa Fluor 405, Abcam ab 175665, 1:200; Goat anti-mouse IgG, Alexa Fluor 647, ThermoFisher A32728, 1:500) were suspended in 1% BSA and incubated with the filters for 2 hours at 37° C. VE-cadherin After fixation, permeabilization, and blocking, the primary antibody (Goat anti-human polyclonal VE-cadherin antibody, R&D Systems AF938, 1:13) was suspended in 1% BSA and incubated with the inserts for 2 hours at 37° C. Von Willebrand Factor. After fixation and blocking, the primary antibody (Rabbit anti-human polyclonal, Abcam ab6994, 1:200) was incubated with the filters in 1% BSA for 1 hour at 37° C. Then the secondary antibody (Goat anti-rabbit polyclonal, ThermoFisher A-21245, 1:200) was incubated with the inserts in 1% BSA for 1 hour at 37° C. For this stain cells were intentionally not permeabilized so the study could see externally released VWF. Epifluorescence images were taken using Leica DMI-8 or EVOS. Confocal images were taken at Georgia Tech's Optical Microscopy Core using a PerkinElmer UltraVIEW VoX spinning disc confocal microscope using a 40× (numerical aperture 1.3) or 60× (numerical aperture 1.49) objective.

Histology. Histology was performed in the Parker H. Petit Institute's histology core. Inserts were fixed (Image-iT™) and embedded in OCT so that the filters are perpendicular to the cutting angle. Blocks were sectioned at 10 μM thickness, stained with H&E, and imaged on Leica DMI-1 with a color camera.

Permeability Assays. Fluorescein sodium salt (Sigma-Aldrich, F6377) was diluted to 30 μM in ALI media. 200 μL media was added to the bottom chamber, and 140 μL media with tracer was added to the top chamber. 50 μL was sampled and replaced with fresh media every 30 minutes for 2 hours. Sample fluorescence was measured in black-walled 96-well plates against a standard curve to determine the mass of tracer in the bottom chamber at each timepoint. Net tracer mass was calculated by accounting for the lost sample at each time point. Permeability was calculated using Equation 1:

$${P}_{a p p} = ( \frac{d C}{d V} ) \times V / ( A {C}_{0} ) \qquad ( Eq . 1 )$$

Where dC/dV is the slope of a linear fit to the concentration vs. time plot, V is the volume of media in the receiver plate (200 μL), A is the surface area of the membrane (0.143 cm²), and Co is the concentration of NaFL added in the top chamber (30 μM).

Viral Exposure. Influenza A virus, subtype H1N1, strain A/Puerto Rico/8/1934 (NCBI:txid211044) (PR8) was provided by Nick Heaton's laboratory at Duke University School of Medicine. Human coronavirus oC43 (HCoV-OC43) was provided by Rabindra Tirouvanziam's lab at Emory University. For infection experiments, PR8 and OC43 were diluted to the desired MOI in cell culture media. Transwell receiver plates were prepared with 200 μL/well of virus-laden cell culture medium. The Transwell insert plate was placed into the virus-loaded receiver plate and incubated at 37° C., 95% humidity, 5% C02 for 1 hour. Following this incubation, the Transwell receiver plate was moved back to an empty receiver plate to return to ALI without rinsing. The exposed cells were further incubated at 37° C., 95% humidity, 5% C02 until the specified endpoint (24, 48, or 72 hours).

Cytokine quantitation. Cell culture supernatants were collected at specified intervals. IL-6 and IL-8 were quantified with ELISA assays according to manufacturer instructions (Human IL-6 DuoSet ELISA, R&D Systems DY206-05; Human IL-8/CXCL8 DuoSet ELISA, R&D Systems, DY208-05; DuoSet ELISA Ancillary Reagent Kit 2, R&D Systems; DY008.

### Experimental Results and Additional Examples

Example #1—Density-driven, inversion-free underside seeding robustly generates functional air-blood barrier model in high-density well throughput. Small airway epithelium was modeled with the club cell line NCI-H441 cultured on a 96-well Transwell culture insert (Corning) opposite a monolayer of primary human umbilical vein endothelial cells (HUVECs). The epithelium was cultured facing downwards, i.e., on the underside of the membrane, due to technical advantages under ALI conditions. Namely, epithelial cells attached outside the co-culture area were rinsed off during ALI culture (FIG. 1), ensuring that the epithelium had a constant surface area and included only co-cultured cells directly opposite the endothelium. Second, underside epithelial culture provided immediate, visual quality control during ALI, e.g., for faulty wells that cannot hold ALI due to failed seeding or contamination leak quickly and collect media in the plate, while successful wells hold liquid in the top chamber. Finally, the liquid in the underlying plate of the 96-well Transwells can be prone to media wicking between wells even when less than the recommended 235 μL of media was used (here 200 μL), presenting contamination risk. Indeed, it was observed that underside epithelial seeding and ALI culture beneficially reduced well-to-well variability, provided quality control, and minimized cross-contamination risk during ALI culture.

In an aspect of the exemplary method, H441 cells were seeded on the underside of 96-well Transwell inserts without plate inversion or removal of the inserts from the underlying plate by manipulating cell culture media density so that cells float to contact the underside of the membrane (FIG. 1). H441 cells were suspended in a cell culture medium of density 1.16 g/mol, a slightly greater density than the cells. The density was experimentally determined to be the smallest that ensures that the majority of cells were floating for the entire attachment period of 2 hours. The dense medium comprised a homogeneous mixture of 50/50 v/v % cell culture media and OptiPrep (STEMCELL™ Technologies), a commercially available solution of 60% w/v iodixanol in water with a density of 1.3 g/mL that is typically used for density-gradient cell separation. The homogeneous, dense cell culture solution caused H441 cells to float and contact the underside of the Transwell membrane, where they adhered and formed a monolayer over a 2-hour period (FIG. 1). After attachment, the dense medium was diluted by adding a regular cell culture medium to give an approximately 1.7:1 ratio of normal to dense media. Following 24 hours, the endothelial cell layer was seeded on the top of the membrane to form a bilayer co-culture model of the small airway-capillary barrier. Notably, the underside seeding method was automatable, and the study performed 96-well seeding using the commercially available Viaflow-96 liquid handling system.

It was observed that the exemplary epithelial seeding method consistently generated monolayered, confluent epithelium with co-cultured endothelium that reached an average peak TEER in co-culture of 521 Ω·cm² on Day 7 of ALI (S.D. 208.35, 95% CI 481.88-560.26) and sodium fluorescein (NaFL) permeability of 7.04e-6 cm/s (S.D. 1.63e-5, 95% CI 1.53e-6-1.25e-5). These barrier function values are consistent with similar reported Transwell co-culture models. Despite all wells typically reaching confluence on both sides of the membrane (observation by fluorescence microscopy of GFP- and RFP-expressing cell layers), some variation in TEER was consistent with other models. However, almost all wells (average of 93/96 wells, or 97%) in every experiment reached the threshold for acceptable barrier function, defined as 285 Ω·cm² (Table 1). This threshold was determined through the observation that an epithelial-endothelial co-culture would always be confluent if its TEER was greater than 250 Ω·cm². Below this value, some wells were not completely confluent. This quality control metric showed quantitatively that wells were confluent and possessed barrier function after seven days of ALI culture.

TABLE 1

| | Met | Total | % |
|---|---|---|---|
| Run | Standard | Seeded | Success |
| A | 91 wells | 96 wells | 94.8% |
| B | 93 wells | 96 wells | 96.9% |
| C | 93 wells | 96 wells | 96.9% |
| D | 95 wells | 96 wells | 99.0% |
| | | AVG | 96.9% |
| | | STDEV | 1.5% |
| | | 95% CI | 2.0% |

Example #2 Bilayer co-culture exhibits polarization and differentiation of epithelial cells in co-culture with primary endothelium. NCI-H441 cells in co-culture with HUVECs were differentiated at an ALI in a serum-free medium containing an Ultroser™ G (Sartorius) serum substitute to promote polarization. After 5 days of ALI, NCI-H441 showed robust expression of the tight junctional proteins ZO-1 and E-cadherin (FIG. 2). The epithelium also demonstrated polarization, shown by the localization of ZO-1 at the air interface with E-cadherin lining cell-cell interactions. The co-cultured endothelium robustly expressed the adherens junctional protein VE-cadherin. In co-culture, the cells showed synergistically elevated barrier function demonstrated by high TEER and low permeability. In co-culture, TEER peaked at day 7 for an average maximum value of 521 Ω·cm² (S.D. 208.35, 95% CI 481.88-560.26) and permeability of NaFL reached 7.04e-6 cm/s (S.D. 1.63e-5, 95% CI 1.53e-6-1.25e-5). Comparatively, NCI-H441 cell monoculture TEER peaked on day 7 at 226.99 Ω·cm² (S.D. 7.15, 95% CI 228.63-225.34) and HUVEC monoculture peaked on day 10 at 30.35 Ω·cm² (S.D. 3.12, 95% CI 28.0-32.7). Epithelial-endothelial co-culture, in comparison to NCI-H441 cell monoculture, demonstrated statistically elevated TEER (p<0.001, student's t-test, heteroscedastic) for the entire culture period from days 0-10 (See Example 2).

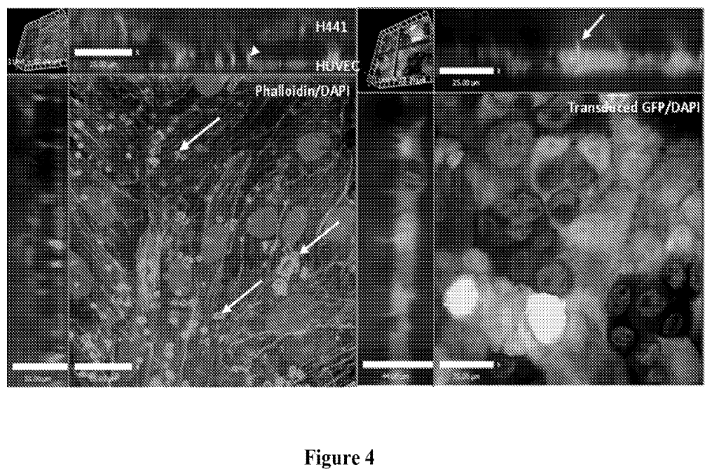

Further, co-culture TEER was also statistically greater than the sum of the epithelial and endothelial monolayers at every day measured (See Example 2). This indicated that culturing the epithelium and endothelium together for the duration of the air-liquid differentiation period can enhance the development of epithelial barrier strength. Alternatively, the increased TEER can be caused in part by the phenomenon of NCI-H441 cells growing into the pores to contact the endothelium (FIG. 4). This epithelial growth into the pores has been observed in similar pulmonary epithelial-endothelial co-culture models cultured on microporous inserts. This interaction is considered to enhance epithelial-endothelial communication in a physiologically relevant manner.

Example 3—Epithelial exposure to viral and bacterial mimics induces endothelial inflammation and barrier loss. Exogenous stimuli such as pathogens can initiate systemic pathophysiology through the propagation of epithelial insult to the endothelium, and the resulting communication of inflammatory signals into the bloodstream where they can travel systemically. To demonstrate the utility of the exemplary model for studying this phenomenon, the study tested if the exemplary air-blood barrier model can recapitulate the transfer of inflammatory signals from the epithelium to endothelium during epithelial exposure to viral determinants and live viral pathogens. The study first exposed the epithelial side of the bilayer to the viral infection mimic polyinosinic:polycytidylic acid (poly(I:C)) for 10 minutes, and showed that the endothelium immediately exocytosed von Willebrand factor (VWF), a pro-coagulant macrostructured glycoprotein that assembles on the endothelial surface in response to inflammatory stimuli to aggregate platelets and attract immune cells. Such immediate VWF release has been reported previously upon direct stimulation of the endothelium with poly(I:C).

To demonstrate barrier function in response to a viral or bacterial mimic, H441 cells in co-culture were exposed to poly(I:C) or lipopolysaccharide (LPS, component of gram-negative bacterial membranes) at 2 doses each for 24 hours. LPS 1 (1 μg/mL) did not induce an increase in permeability, while LPS 2 (20 μg/mL) did (FIG. 2). Permeability of NaFL was measured after 24 hours of poly(I:C). Doses 1 and 2 (1 and 10 μg/mL) induced a permeability increase. Permeability of the control bilayer co-culture was 7.04×10-6 cm/s+0.163×10-6 cm/s measured by NaFL over a 3-hour period. In the exposed group, 2 of the 4 exposure conditions resulted in statistically significant increases in permeability compared to the controls that received no exposure and remained at ALI; n=3 per condition; n=4 for controls. Analysis was performed with a 2-way ANOVA test with post hoc Tukey's t-test (GraphPad Prism).

Example #4—Epithelial Viral Exposure Induces Dose-Dependent Inflammatory Signals in Epithelial-Endothelial Co-culture. The study exposed the epithelial H441 cell layer to influenza A virus (subtype H1N1, strain A/Puerto Rico/8/1934) and human beta-coronavirus (HCoV-OC43) in cell culture media for one hour before returning the epithelium to ALI. This allowed the virus to attach while maintaining the epithelium at ALI over the 3-day influenza. Infection of the epithelium by influenza A virus and human beta-coronavirus resulted in dose-dependent endothelial production of leukocyte chemoattractant IL-8 after 72 hours, indicating that epithelial infection can lead to endothelial induction of the appropriate inflammatory response. Of note, after 72 hours, none of the wells had lost ALI (no media leaked through to the bottom well). This system configuration demonstrates both the feasibility of detecting MOI-dependent responses in the co-culture system and the capability to enable parallel screening of a multiplicity of conditions with one plate.

Indeed the exemplary system and method exploited density-driven cell buoyancy to enable underside attachment without inversion. As an initial demonstration of this underside seeding method, the study constructed a co-culture model of the small airways (bronchioles). This region of the lung, which is close to the alveoli, is heavily involved in the mediation of inflammatory responses during toxin and pathogen exposure. Successful barrier maintenance in this region is critical to prevent acute lung injury from developing after insults or infection. The study showed that upon stimulation of the epithelial side of an engineered air-blood barrier with bacterial or viral insults, the apposing endothelium exhibited prothrombotic (vWF release) and proinflammatory (IL-8 secretion) responses. The many wells available for testing conveniently allowed the comparison of the effect of different viruses, MOI, and time points.

More broadly, epithelial-endothelial tissue barriers can be employed to control access to the bloodstream at a variety of tissue sites, including the respiratory tract and the mucosal barriers of the nasal passage, intestine, and eyes. The exemplary seeding method can thus be applicable to models emulating these other mucosal sites as well. Different cell types may require tuning of the ratio of Optiprep to cell culture media. Here this study used a 50/50 vol/vol split because it was well tolerated by the epithelial cells and resulted in near 100% flotation during the 2-hour culture period. However, the 50/50 setup resulted in a density of 1.16 g/mL and a relatively increased liquid viscosity that can injure sensitive cell types. Most cells' density was close to 1.05 g/mL, so a greater media:Optiprep ratio can still prove effective for more delicate cell types that need more media and lower viscosity to tolerate this method.

In conclusion, bilayer co-culture enhances the physiological relevance of many high-throughput tissue barrier models. However, the broader adoption of bilayer co-culture systems had been hampered by the difficulty of seeding cells on the underside of the membrane, and prior procedures required the inversion of the membrane inserts and were particularly difficult in large plate configurations, e.g., featuring 96 or more scales. This instant study demonstrated that a robust co-culture model of the small airway epithelial-endothelial barrier can be produced at the 96-well scale using a robotic liquid handler-compatible procedure that circumvents the need for plate inversion during cell seeding by using high efficiency (>97%) attachment by density-driven cell flotation instead. The instant method can be employed to enhance the convenience of high-throughput co-culture and increase physiologic relevance of tissue barrier models for high-throughput screening.

### Example 2. Standard Operating Procedures: Inverted Seeding and Air-Liquid Interface Coculture of H441/HUVEC in Large-Well Transwell HTS

Description: A method is now described to grow a cocultured model of the air-blood barrier with H441 epithelial cells and primary HUVECs at an air-liquid interface. The model can produce well-differentiated H441 monolayers with high TEER (350-400 ohms*cm²), tight junctional protein expression (ZO-1 and e-cadherin), adherens junction protein VE-cadherin, and expression of ACE2 (confirmed by RT-PCR) and TMPRSS2 (confirmed by immunofluorescence staining). The model can be optimized for use 5 full days after the initiation of air-liquid interface culture.

Schedule Summary

TABLE 2

| Day | Step | Media | Instructions | Notes |
|---|---|---|---|---|
| −1 | Collagen coat | | | Dry in the BSC |
| | | | | OVERNIGHT for best |
| | | | | results |
| 0 | Seed H441 | TOP: nothing | | Optiprep at 37° C. |
| 0 hrs | | BOT: 85 uL 50/50 | | Use new tips for every |
| | | vol % Opti/ | | aspiration because |
| | | RPMI-P | | volumes quickly get off |
| | | | | mix one aliquot and |
| | | | | sample ONE well |
| | | | | before you do the whole |
| | | | | plate and check on |
| | | | | EVOS |
| 0 | Supplement | TOP: 75 uL | Do not remove old | Important- 2-3 hours |
| 2 hrs | media | RPMI-P | media or move | and not longer |
| | | BOT: 150 uL | inserts to a new | |
| | | RPMI-P | plate. | |
| 1 | Seed | TOP: 100 uL | Use a new bottom | After seeding, check on |
| | HUVEC | VCBM + S | plate with fresh | EVOS that cells are |
| | | BOT: 200 uL | media. | evenly distributed on |
| | | RPMI-P | | the membrane. |
| 2 | MC-LL | TOP: 150 uL | Remove & replace | Viaflow for top well |
| | | VCBM + S | RPMI in bottom | MCs- B plate height |
| | | BOT: 200 uL | plate. | 82.5 mm. |
| | | RPMI-P | | |
| 3-10 | MC-ALI | TOP: 200 uL ALI | Using Viaflow, | If H441/HUVEC aren't |
| (Daily) | | BOT: Air | remove top media | confluent by D3 they |
| | | | and replace with 150 | might grow in during |
| | | | uL fresh ALI media. | ALI so you can try |
| | | | | lifting if TEER is > |
| | | | | 1000. |
| | | | Use a new bottom | confluent wells will |
| | | | plate. | have TEER >= ~3000 |
| | | | | ohms (uncorrected) |
| | | | | 5 days post-ALI, TEER |
| | | | | will peak ~3000-4000 |
| | | | | ohms (uncorrected), |
| | | | | typically on Day 8. |
| | | | | 7-8 days post-ALI, |
| | | | | HUVECs will start |
| | | | | losing confluence. |

Media Compositions

TABLE 3

| | Abbreviation | Media composition |
|---|---|---|
| H441-GFP: | RPMI + P | RPMI-1640 |
| T-75 | | 1 × P/S |
| | | 10 v/v % FBS |
| | | 1.5 ug/mL puromycin |
| H441-GFP: | RPMI-P | RPMI-1640 |
| Transwell liquid- | | 1 × P/S |
| liquid | | 10 v/v % FBS |
| HUVEC-RFP: | VCBM + S | VCBM (ATCC) |
| T-75 and | | Endothelial growth kit |
| Transwell liquid- | | with VEGF (ATCC) |
| liquid culture | | |
| Transwell | ALI | 50 v/v % VCBM + S |
| air-liquid | | EXCEPT FBS |
| interface co- | | 50 v/v % RPMI-1640 |
| culture | | 1:50 Ultroser G (Sartorius) |
| | | aliquoted in −20° C. |
| | | 1:100 P/S (1×) |

Day −1. Collagen coating.

Amount: Two 96-well plates' worth of inserts, scale as needed.

TABLE 4

| | | | | | Product |
|---|---|---|---|---|---|
| Amount | Concentration | Material | Notes | Vendor | Number |
| 100 uL | ~3 mg/mL | Rat Tail | 4° C. | Corning | 354236 |
| | | Collagen Type I | | | |
| 10 mL | 60 v/v % | EtOH in mQ H2O, | 4° C. | Made in | |
| | | pH < 7 | | lab | |

Materials and Methods

TABLE 5

| | | | Catalog |
|---|---|---|---|
| Name | Location | Vendor | # |
| HTS Transwell-96 Permeable | | Corning | CLS3386 |
| Support with 3.0 μm pore | | | |
| polycarbonate membrane, | | | |
| TC-treated, sterile | | | |
| HTS Transwell ®-96 Receiver | | Corning | 3382 |
| Plate, Clear, Sterile | | | |

Example Process

TABLE 6
In BSC: In ice, bring in collagen and pre-cooled (4° C.) 60% EtOH. Bring in a Transwell insert
plate in a receiver plate.
In BSC: On ice, dilute collagen 1:100 to ~30 ug/mL. (outside BSC) Vortex to mix. (A) Pipette
collagen slowly to ensure you get the correct volume. Ensure that the pipette does not touch
anything inside the collagen container. The pipette isn't sterile, only the tip is sterile. (B) Add
collagen to already-prepared volume of EtOH on ice. Pipette up and down a few times to
dissolve all collagen off the pipette tip into the EtOH. (C) Make excess solution (e.g., for 25
wells, make for 30, etc). Add 100 uL collagen to 10 mL EtOH (e.g., for a 2 96-well plates at
30.3 uL/well). (D) Use the yellow/gray multichannel because it's the right volume range - the
blue ones are not in range.
In BSC: Pipette 30.3 uL/well onto inverted Transwell inserts. Allow to dry in the BSC for at
least 4 hours (Preferably overnight) with the hood open to maintain sterility.
(A) Invert the Transwell plate + lid. Remove the receiver plate leaving the insert tray on its lid.
Transwells will now be upside down resting atop the lid of the 96-well plate. Ensure to maintain
sterility of the empty 96-well plate plus lid to be used to house Transwells once they have been
seeded with epithelial cells.
(B) Use a multichannel media reservoir and the 8-well small-volume multichannel pipettor:
the gray one with yellow accents, it is labeled with a range of 5-50 uL. Use 10-100 uL barrier
tips.
(C) Add pre-vortexed collagen solution to the reservoir, and slosh around to mix. Carefully
pipette 30.3 uL per well on each membrane. Do not touch the membrane with the tips. Just get
close enough to deposit the liquid. Some liquid may run down the side. Try to avoid but it's
not necessary to completely prevent. Be careful that the collagen solution does not drip down
the side of the Transwell and avoid moving Transwells during the coating procedure.
(D) Leave the inserts in the center of the hood to dry with the shield open for at least 4 hours.
(E) It can be determined that the inserts are dry when the inserts become flat and clear. If
they're still wrinkly, they aren't dry yet.
After the Transwells have dried, they can be used immediately or stored at room temperature
for up to a week provided they are returned to the 96 well plate and sterility can be maintained
(store them in the hood).

Day 0. H441 Inverted Seeding

Amount: 2 96-well plates' worth of inserts, scale as needed

TABLE 7

| | | | | | | Product |
|---|---|---|---|---|---|---|
| Amount | | Concentration | Material | Location | Vendor | Number |
| 2 | flasks | 70-90% | NCI-H441 T-75 | Incubator | ATCC | ATCC ® |
| | | confluence with | | | | HTB-174 ™ |
| | | smooth edges | | | | |
| 20 | mL | 1× | PBS, sterile, 37° C. | 4° C. | | |
| 100 | mL | | RPMI-P | 4° C. | Made in lab | |
| 12 | mL | | OptiPrep ™ | 4° C. | STEMCELL | 07820 |
| | | | Density gradient | | Technologies | |
| | | | medium | | | |

| | | | |
|---|---|---|---|
| 2 × 2-mL | 0.05%/0.02% | Trypsin/EDTA | −20° C. |
| aliquots | | | |

Materials

TABLE 8

| Name | Notes | Vendor | Catalog # |
|---|---|---|---|
| Collagen-coated HTS 96-well | Incubator | Corning | |
| plate (Previous protocol) | | | |
| DMI-1 (Light microscope | | Leica | |
| with 5 × objective) | | | |
| EVOS Epifluorescence microscope | | Life | |
| with 2 × & 4 × obj | | Sciences | |
| EVOS GFP filter cube | | Life | |
| | | Sciences | |
| EVOS RFP filter cube | | Life | |
| | | Sciences | |

Example Process

TABLE 9
(1) 20 minutes before start: Warm up reagents, new flasks, and plate prior to beginning the
protocol.
(2) Warming step is critical for proper seeding. Without warm Opti & media, cells won't
attach well. (A) Aliquot and place in bead bath: (i) 100 mL RPMI-P; (ii) 20 mL PBS; (iii) 12
mL Optiprep; and (iv) 2 aliquots 0.05% Trypsin. (B) Prepare two T-75 flasks with RPMI + P,
20 mL per T-75, and place them in the incubator. (C) Place the collagen-coated plates to be
seeded in the incubator.
(3) In BSC: Passage H441s:
(A) Aspirate old media from the T-75 s.
(B) Rinse each with 10 mL warm PBS and wait 3 min.
\textcircled{C}Aspirate PBS.
(D) Add 2 mL 0.05% trypsin-EDTA (1 aliquot) and swirl around the flask to cover cells
evenly.
€ Observe cells on the DMI-1 until they are rounded and begin detaching.
(F) When they appear fully rounded, swirl the flask and tap on your palm to lift. Do not tap on
the bench, it can be too rough on cells and may create clumps.
(G) When > 95% of cells are lifted on DMI-1 inspection, return to the BSC.
For each flask separately:
(H) Tilt the flask to collect cells in the bottom corner. Use a 5 mL serological to pipette cells
up and down to break up clumps before trypsin inactivation.
(I) Using a 10 mL serological, add 5 mL RPMI-P to inactivate trypsin: (i) Tilt the flask and
pipette along the bottom of the flask to detach cells and flow them to the corner; (ii) Pipette up
and down several times to disrupt clumps; and (iii) Collect cells and place in 15 mL Falcon
tube.
(J) Do not combine multiple flasks in one tube for centrifugation OR counting.
(K) Centrifuge to pellet cells for 5 min at 200 g, 25° C. During the centrifuge, prepare for cell
counting and seeding: (i) Bring in P100 pipette tips; (ii) Bring in 0.5 mL Eppendorf tubes; (iii)
Turn on the cell counting computer & open the software; (iv) Peel the plastic off the counting
chamber; and (v) Bring the newly warmed up T-75s into the hood & label with initials, cell
type, passage number & date.
(L) In BSC: After centrifuge, aspirate the supernatant and resuspend cells in 1 mL RPMI-P.
Pipette the top of the cells to gently disrupt the pellet. Try not to lift up or aspirate the pellet.
(M) Add 2 more mL RPMI-P to dilute cells for accurate counting. Cell counter precision can
be greatly reduced at high cell concentration (>2e6 cells/mL)- personal observation.
(N) Count H441s several times by shifting the counting chamber's position. Choose the lowest
number as the cell density and write this number down. Repeat for each flask.
(O) Seed the two fresh T-75's with 1e6 cells each and return to the incubator.
(P) Dilute each tube of cells to 1.18e6 cells/mL by adding the appropriate volume of RPMI-P.
Tilt/rock the tube to mix.
(4) Prepare for seeding.
(A) Preparation: (i) Bring in the 12-well multichannel pipettor, P200 tips, and reservoirs; (ii)
Bring in a 0.5 mL Eppendorf tube and set the P200 pipettor to 85 uL; (iii) Turn on the Evos,
clean the stage, and set the vessel to 96-well plate, the objective to 2×, and the channel to GFP;
(iv) Bring in the pre-warmed Optiprep; and (v) Bring in the collagen-coated, pre-warmed plate
(B) Test cell seeding density with one well: (i) Re-mix your cell suspension by rocking the
closed tube back and forth 5-10 times; (ii) Collect 85 uL of cell suspension and add to the 0.5
mL Eppendorf; (iii) Collect 85 uL Optiprep and add to the 0.5 mL Eppendorf. Pipette up and
down to mix thoroughly until no swirling is observed; and (iv) Collect 85 uL of mixed solution
and add to the bottom right corner well (lower chamber) using the small access hole; (v)
Inspect the seeding by looking at cell distribution on EVOS. If it looks bad, figure out why.
Use the reference pictures (located later in this document) to compare with the resulted
seeding. Sometimes the cells don't distribute evenly, so it may be necessary to gently shake the
plate in the XY direction to distribute (if Z is up/down, XY is left/right)
(5) If the test seeding is successful, continue to seed the rest of the plate: (i) Combine an equal
volume of cell suspension (already adjusted to 1.18e6 cells/mL) and Optiprep in a new Falcon
tube. Pipette up and down to mix a little, then rock the tube back and forth until it looks
homogeneous. (ii) Prepare your multichannel pipettor, get tips ready, place an empty reagent
reservoir, and take the lid off the collagen-coated plate. (iii) Re-mix the opti/cell mixture by
rocking, then pour the solution into the reagent reservoir. Immediately begin seeding rows,
using the 12-channel pipettor set to 85 uL. Do not reuse tips for more than 3 rows. Ideally, use
new tips for each row. Volumes could be wrong if tips are re-used excessively. Work quickly.
Cells may start floating in the reagent reservoir if too slow, and seeding will be uneven. If
there is a need to stop or process is proceeding slowly, re-mix the solution using a serological
pipettor, pipette up and down multiple times.
(6) After seeding all wells, look at them on EVOS to see if they're distributed evenly in the
well, if any wells have any issues that you can fix. See troubleshooting section below for
details.
(7) Once wells look good, place the plate in the incubator for 2 hours.
(8) After 2 hours, add 75 uL RPMI to the top chamber and 150 uL to the bottom and return the
plate to the incubator for 12-16 hours.
(9) After this time you can move the inserts to a fresh plate with 200 uL new RPMI-P on the
bottom and seed HUVECs.

Day 1. HUVEC Seeding & Liquid-Liquid Media Change

Amount: 2 96-well plates' worth of inserts, scale as needed

TABLE 10

| | | | | | | Product |
|---|---|---|---|---|---|---|
| Amount | | Concentration | Material | Notes | Vendor | Number |
| 2 | flasks | 70-90% | HUVEC-RFP | Incubator | ATCC | ATCC ® PCS- |
| | | confluence | in T-75 s | | | 100-013 ™ |
| 75 | mL | | VCBM + S | 4° C. | ATCC | |
| 40 | mL | 1× | PBS, sterile, 37° C. | 4° C. | | |

| | | | | | |
|---|---|---|---|---|---|
| 2 × 2-mL | 0.05%/0.2% | Trypsin/EDTA | −20° C. | | |
| aliquots | | | | | |
| 2 × 5-mL | | Trypsin neutralizing | −20° C. | Lonza | CC-5002 |
| aliquots | | solution (TNS) | | | |

Materials

TABLE 11

| Name | Vendor |
|---|---|
| DMI-1 (Light microscope | |
| with 5× objective) | |
| EVOS (Epifluorescence | Life Sciences |
| microscope with 2× & | |
| 4× obj) | |
| EVOS GFP filter cube | Life Sciences |
| EVOS RFP filter cube | Life Sciences |
| Tabletop Centrifuge | |

Example Process

TABLE 12
(I) 20 minutes before start: Warm up reagents, new flasks, and plate prior to beginning the
protocol: (A) Aliquot and place in bead bath: (i) 35 mL VCBM+S; (ii) 40 mL PBS; (iii) 2 2-
mL aliquots 0.05% Trypsin; and (iv) 2 5-mL aliquots Trypsin Neutralizing Solution (TNS);
(B) Prepare two T-75s with VCBM+S (20 mL per T-75), and place in incubator; (C) Prepare
2 new 96-well receiver plates with 200 uL/well RPMI+P and warm in the incubator.
(II) In BSC: Passage HUVECs:
(1) Rinse with PBS: (A) Aspirate old media from T-75s; (B) Rinse with 10 mL warm PBS
and wait 3 min; (C) Aspirate PBS; (D) Rinse with 10 mL warm PBS and wait 3 min; and (E)
Aspirate PBS.
(2) Add 2 mL 0.05% trypsin-EDTA (1 aliquot) and swirl around the flask to cover cells
evenly.
(3) Observe cells on the DMI-1 until they are rounded and begin detaching.
(4) When they appear fully rounded, swirl the flask and tap on your palm to lift. Do not tap
on the bench, it can be too rough on cells and may create clumps.
(5) When >95% of cells are lifted on DMI-1 inspection, return to the BSC.
For each flask separately:
(6) Tilt the flask to collect cells in the bottom corner. Use a 5 mL serological to pipette cells
up and down to break up clumps before trypsin inactivation.
(7) Using a 10 mL serological, add 1 aliquot TNS to inactivate trypsin.
(8) Tilt the flask and pipette along the bottom of the flask to detach cells and flow them to the
corner.
(9) Pipette up and down several times to disrupt clumps
(10) Collect cells and place in 15 mL Falcon tube. Do not combine multiple flasks in one tube
for centrifugation OR counting.
(11) Centrifuge to pellet cells for 5 min at 200 g, 25 C. During the centrifuge, prepare for cell
counting and seeding: (A) Bring in P100 pipette tips; (B) Bring in 0.5 mL Eppendorf tubes;
(C) Turn on the cell counting computer & open the software; (D) Peel the plastic off the
counting chamber; (E) Bring the newly warmed up T-75s into the hood & label with initials,
cell type, passage number & date.
(12) In BSC: After centrifuge, aspirate the supernatant and resuspend cells in 2 mL VCBM+S
using P1000. (A) Pipette 1 mL on the top of the cells to gently disrupt the pellet. Try not to
lift up or aspirate the pellet. (B) Add 1 more mL RPMI-P to dilute cells for accurate counting.
Do not skip dilution. Cell counter precision can be greatly reduced at high cell concentration
(>2e6 cells/mL)—personal observation.
(13) Count cells several times by shifting the counting chamber's position. Choose the lowest
number as the cell density and write this number down. Repeat for each flask.
(14) Seed the two fresh T-75's with desired cell density and return to the incubator. HUVEC
cell density depends on when the cells will need to be passaged again. Density can range
from 2500 cells/cm² (~4 days to passage) up to 5000 cells/cm² (~24-48 hrs to passage).
(15) Dilute each tube of cells to 80,000 cells/mL by adding the appropriate volume of
VCBM+S. Tilt/rock the tube to mix.
(16) Prepare for seeding: (A) Bring in the 12-well multichannel pipettor, P200 tips, and
reservoirs; (B) Bring in a 0.5 mL Eppendorf tube and set the P200 pipettor to 100 uL; (C)
Turn on the Evos, clean the stage, and set the vessel to 96-well plate, the objective to 2x, and
the channel to RFP; and (D) Bring in the H441-seeded plate.
(17) In BSC: Change bottom plate to the prepared plate from Step 1 that has fresh media.
(18) In BSC: Test cell seeding density with one well: (A) Re-mix cell suspension by rocking
the closed tube back and forth 5-10 times; (B) Collect 100 uL of cell suspension and add to
the bottom right corner well (upper chamber); (C) Inspect the seeding by looking at cell
distribution & density on EVOS. If it looks bad, figure out why. Use the reference pictures
(located later in this document) to compare with the seeding; and (D) If the cells do not
distribute evenly, gently pipette the upper chamber media up/down a few times to re-mix the
cells so they seed evenly.
(19) In BSC: If the test seeding is successful, continue to seed the rest of the plate: (A)
Prepare your multichannel pipettor, get tips ready, place an empty reagent reservoir, and take
the lid off the H441-seeded plate; (B) Using the Viaflow, remove media from the top
compartment with the B-plate height set to 82.5 mm; (C) Re-mix the HUVECs by rocking the
closed Falcon tube. If a pellet is visible, resuspend it with the P1000; and (D) Immediately
after mixing, pour the solution into the reagent reservoir. Immediately begin seeding rows,
using the 12-channel pipettor set to 100 uL. Note: Do not reuse tips for more than 3 rows.
Ideally, use new tips for each row. Volumes could be wrong if tips are re-used excessively.
Work quickly. Cells may start floating in the reagent reservoir if too slow, and seeding may
be uneven. If there is a need to stop or the process is proceeding slowly, re-mix the solution
using a serological pipettor, pipette up and down multiple times.
(20) After seeding all wells, look at them on EVOS to see if they're distributed evenly in the
well, if any wells have any issues that you can fix. See troubleshooting section below for
details.
(21) Once wells look good, place the plate in the incubator until Day 2.

Day 2. Liquid-Liquid Media Change

Example Process

TABLE 13
(1) In BSC: prepare a new bottom plate with 200 uL/well RPMI-P (doesn't need to be warm).
Incubate the plate at 37° C. for at least 15 min before protocol start.
(2) In BSC: Using the Integra Viaflow-96, remove media from the top wells with B-channel
height set to 82.5 mm. Alternatively, use a multichannel pipettor to manually remove media.
Keep the pipettor at a 15 degree angle so you don't puncture the wells.
(3) In BSC: Using a multichannel pipettor or Viaflow-96, add 100 uL/well VCBM+S to top
wells.
(4) In BSC: Carefully move the insert plate from the old receiver to the fresh pre-warmed
receiver plate. Move slowly when taking the inserts out and putting them back. The cells are
not yet differentiated and will detach easily if stressed. The fluid meniscus should not tear
them off if you're delicate and slow.

Day 3: Lift to Air-Liquid Interface Culture

Example Process

TABLE 14
By Day 3 if everything goes well, both layers will be confluent.
If there are small holes in the H441 layers they will usually close up during ALI.
Significant deficiencies (>=30% of the well) will probably not.
Do not lift to ALI if TEER is below 600 because they will probably not be able to hold
liquid (it'll drip through to the bottom).
Confluent bilayers will have TEER >= about 2500 ohms. Below this they are most likely
not filled in.
(1) In BSC: Move the inserts to an empty receiver plate.
(2) In BSC: Remove as much media from the top wells as possible with a multichannel
pipettor or use the Viaflow.
(3) In BSC: Add pre-warmed ALI media to the top channels up to the top of the well (about
140 uL).
(4) Replace in the incubator.
On day 3, TEER matypeak at 3500-4500 ohms (uncorrected). On Day 4 (first 24 h following
switch to ALI), TEER may drop to 400-600 ohms (uncorrected). It should take 3-5 days to
climb back up to a peak around 2500 ohms. Day 7 or 8 is the optimal time for assay usage
(5-6 days ALI).

Day 3-8: Maintenance at ALI.

Example Process

TABLE 15
Media change at around the same time every day. Cells cannot tolerate 48-hour increments for
media change, so it must be done daily.
(1) In BSC: Remove as much media from the top wells as possible with a multichannel
pipettor or use the Viaflow with B-plate height at 82.5 mm.
(2) In BSC: Add pre-warmed ALI media to the top channels up to the top of the well (140 uL).
(3) Replace in the incubator.
Maintain plate at ALI until day 7-8 for optimal assay use. Stains for ZO-1, e-cadherin, VE-
cadherin, TMPRSS2, and F-actin are on day 6-8. Maximum TEER at 5^{th} day of ALI.
Therefore Day 7-8 is the best time to use the plate.

Accessory Protocols

A) 60% EtOH

Amount: ten 96-well plates' worth of EtOH, scale as needed.

TABLE 16

| Amount | | Conc | Material | Vendor | Product # |
|---|---|---|---|---|---|
| 42.9 | mL | 70% | Ethanol, 70% Solution, | Fisher | BP8203- |
| | | | Molecular Biology Grade, | BioReagents ™ | 1GAL |
| | | | Denatured, Fisher | by Fisher | |
| | | | BioReagents ™ | Scientific | |
| 7.1 | mL | | Milli-Q water | | |

Example Process

TABLE 17
(A) At bench: Dilute 70% w/w EtOH (directly from the bottle, not from the EtOH tub in cell
culture) to 60% v/v with Milli-Q water. Vortex to mix. This has to be 70% EtOH. Other %
EtOH doesn't have the correct pH.
(B) In BSC: Sterile filter with 0.22 um pore size.
(C) Place on ice or in 4° C. fridge for at least 30 min prior to starting collagen coating
protocol.

B) Air-Liquid Interface Media

Amount: 50 mL (scale as needed; recommended 250 mL at a time for 2 full 96-well plates).

TABLE 18

| | | | | | Product |
|---|---|---|---|---|---|
| Amount | Conc | Material | Notes | Vendor | Number |
| 25 mL | — | VCBM+S | 4° C. | ATCC | ATCC ® PCS- |
| | | (NO FBS) | | | 100-030 ™ |
| 25 mL | — | RPMI-1640 | 4° C. | ATCC | ATCC ® 30- |
| | | Base medium | | | 2001 ™ |
| 1 mL | | Ultroser G | −20° C. | Sartorius | 15950-017 |
| | | | 1 mL | | |
| | | | aliquots | | |
| 1 | 5-mL | 100× Penicillin/ | −80° C. | Corning | |

| | |
|---|---|
| aliquot | Streptomycin |

Example Process

TABLE 19
(1) In BSC: Mix 25 mL each RPMI-1640 (base media with no supplements) and VCBM+S
(NO FBS) in a 50 mL Falcon tube, followed by 1 mL aliquot of USG pre-thawed in a 37° C.
bead bath. Larger volumes can be mixed in a 500 mL sterile bottle.
(2) Add Penicillin/streptomycin in a 1:100 ratio to the total solution volume. Exampl: If I
make 250 mL of media, I will add 250/100 = 2.5 mL P/S to the solution.
(3) In BSC: Vortex and swirl around to thoroughly mix the solution.
(4) In BSC: Aliquot into 15 mL falcon tubes for storage at 4° C. up to one month from the
date that you added the endothelial growth kit to the VCBM. Don't store the ALI media in a
larger container than needed. If it gets exposed to air the pH will change. If it turns very pink
don't use it. Store at 4° C. with minimum exposure to extra air.

C) TEER in Transwell HTS-96 with EVOM2 and STX100C96

Amount: 2 96-well plates' worth of inserts, scale as needed.

TABLE 20

| | | | | | | Product |
|---|---|---|---|---|---|---|
| Amount | | Conc | Material | Notes | Vendor | Number |
| 45 | mL | 1× | PBS, sterile, 37° C. | 4° C. | | |
| 5 | mL | 70% | Ethanol | | | |

Equipment

TABLE 21

| Name | Vendor | Catalog # |
|---|---|---|
| EVOM2 0-10 kΩ Range Epithelial | World Precision | Order code: |
| Volt/Ohm Meter | Instruments | EVOM2 |
| Electrode STX100C96 (Manual | World Precision | Order code: |
| Electrode for EVOM2, Use with | Instruments | STX100C96 |
| Coming HTS 96-Well Plate) | | |

Example Process

TABLE 22
(1) Place PBS in incubator to warm up for at least 20 minutes.
(2) Bring EVOM2 and electrode (STX100C96) into the BSC. (A) spray EVOM2 with EtOH
and wipe down. (B) Spray electrode and cord with EtOH and wipe down.
(3) Once electrode is in the incubator, place it in 70% EtOH in a 50 mL conical vial for at
least 10 sec.
(4) Plug electrode into EVOM2 and make sure the switch is set to Ohms, not millivolts
(5) Bring in a paper towel and pen and prepare it to record values. Write today's date and the
plate seed date on the towel for later.
(6) Bring in the plate on an aluminum warming plate.
(7) If at ALI: fill bottom wells with 200 uL/well pre-warmed PBS at 37° C. Do not remove
the inserts to do this, use the small access port. Ensure tips are all the way to the bottom and
dispense slowly to minimize wicking between wells.
(8) Dip electrode in PBS to rinse off alcohol and remove from PBS. You can tap the electrode
to remove excess liquid. Do not dry.
(9) Measure TEER: (a) place electrode in the Transwell making sure the wider base is
positioned inside the well and narrower base is outside the well; (b) Allow the electrode to
balance by itself on the Transwell-disturbing it will change the measurement; and (c) Make
sure electrode is not tilted when it stands on its own. Move around the wire of the electrode
until it balances flat.
(10) Wait several seconds (<10 sec) for a consistent reading to appear and record the number
along with the position of the Transwell. Do not leave in longer than 10 sec. It may not flatten
out in 10 sec but it gets within a +/-100 ohm range. The first two units will even out
(thousands and hundreds place) and record a medium value for the tens and ones place.
(11) Between readings of different wells, you do not have rinse the TEER electrode-it would
be best to clean & rinse between wells but it would take over an hour probably.
(12) After reading all wells remove PBS from ALI wells and put the plate back in the
incubator.
(13) Rinse the electrode prongs in DI water (note-cell culture room giant container is EtOH
NOT water)
(14) Rinse electrode in EtOH
(15) Dry the electrode with a kim wipe and store it in its box
(16) Turn off the EVOM2 and store it.
Maintenance: every 1-2 weeks, soak the electrode overnight in Tergazyme to remove protein
buildup. Following this, soak 5 min in pure Clorox (5% hypochlorite). If the tips still do not
appear silvery you can gently sand with the sandpaper provided from the manufacturer. The
metal layer is very thin so be careful not to sand too aggressively or too often or you will
destroy the electrode. TEER can be sensitive to temperature and fluid volume so these
parameters should be kept as consistent as possible. Measure TEER on a warming plate to
keep temperature consistent.

D) Maintenance of NCI-H441 human adenocarcinoma cell line.

Example Process of Media schedule from thaw

TABLE 23

| | |
|---|---|
| Day 0 | Thaw and plate into T-75 |
| | with 20 mL RPMI+P |
| Day 2 | MC 20 mL |
| Day 4 | MC 30 mL |
| Day 6 | MC 40 mL |
| Day 8 | Passage |

e

TABLE 24

| | |
|---|---|
| e | Passage and plate into T-75 |
| | with 20 mL RPMI+P |
| Day 2 | MC 20 mL |
| Day 4 | MC 40 mL |
| Day 6 | Passage |

Example Process of Making Media

TABLE 25

| Volume | | Conc | Material Name | Notes | Vendor | Catalog # |
|---|---|---|---|---|---|---|
| 500 | mL | | RPMI-1640, hi- | 4° C. | ATCC | ATCC ® 30- |
| | | | glucose, with L- | | | 2001 ™ |
| | | | glutamine | | | |
| 50 | mL | | Fetal bovine | −80° C. | Sigma | |
| | | | serum (FBS) | | | |
| 5 | mL | 100× | Penicillin/ | −80° C. | Aliquoted | 100 mg/mL |
| | | | Streptomycin | | | strep, |
| | | | | | | 100 units/mL |
| | | | | | | pen from |
| | | | | | | manufacturer |
| 10 | uL | 20 | Puromycin | | Aliquoted | |
| | | mg/mL | | | | |

Example Process

TABLE 26
(1) 30 minutes before starting: Warm up FBS, pen-strep, puromycin in 37° C. incubator (Cell
culture room).
(2) e Add 50 mL FBS & 5 mL Pen-strep to the RPMI-1640 and gently rock to mix. Always
pipette liquids in the hood, do not pour.
(3) In BSC: Add 37.5 uL of 20 mg/mL puromycin to 500 mL media for a final concentration
of 1.5 ug/mL.
(4) Store at 4° C. for up to 1 month. Sterile filtration is not needed but can be done once if
sterility is compromised at any point. Do not store media exposed to large surface area of air,
as this will alter the pH as indicated by a change in media color.

ii) Changing H441 Media in T-75 Flasks.

Example Process

TABLE 27

1. 20 minutes before starting: Warm up RPMI in 37° C.
incubator (Cell culture room)
2. In BSC: Aspirate out old media from cells. Tilt the cells to bring
media to the corner and be careful not
to touch the layer of cells. Do not scrape or touch cells
3. In BSC: Slowly pipette 10-15 mL of PBS gently onto the side
of the T75, not directly onto the cells.
4. In BSC: Seal T75 and gently rock back and forth to rinse media
from cells.
5. In BSC: Aspirate out PBS from cells being careful like before
not to touch cells.
6. In BSC: Slowly pipette RPMI gently onto the side of the T75,
not directly onto the cells.
7. Return cells to the 37° C. incubator.

iii) Thawing NCI-H441-GFP:

Example Process

TABLE 28

1. Prepare a T-75 with 20 mL RPMI + P and incubate at least 20 min
prior to beginning protocol.
2. Warm 10 mL RPMI + P and a small beaker of water in 37° C.
bead bath
3. Remove vial from LN2 and delete its row on the inventory
spreadsheet
4. Hold the vial in a 37° C. water bath to thaw the cells
5. When the last crystal is gone use the P1000 to move the cells into
an empty 15 mL falcon tube
6. Rinse the cryovial with 1-2 mL RPMI to remove remaining cells
7. Fill the cryovial to 10 mL with RPMI
8. Centrifuge to collect cells and remove DMSO at
200 g for 5 min, 25 C.
9. Aspirate supernatant, resuspend the pellet and plate all of the
cells in a T-75.

NOTE:
Attrition is usually low enough that you don't need to count before you plate, vials are frozen at 1e6 cells/mL. so you're plating 1e6/75 cm² which is about 13.3k cells/cm².

iv) Passaging NCI-H441-GFP.

Amount: 1 flask.

This protocol may be applied for passaging. Passaging for the purpose of seeding a 96-well plate should follow the protocol, e.g., as described on Day 0 in the detailed protocol.

TABLE 29

| | | | | | Product |
|---|---|---|---|---|---|
| Amount | Conc | Material | Notes | Vendor | Number |
| 1 flask | 70-90% | NCI- | Incubator | ATCC | ATCC ® |
| | confluence | H441 T-75 | | | HTB- |
| | with smooth | | | | 174 ™ |
| | edges | | | | |
| 10 mL | 1× | PBS, sterile, | 4° C. | | |
| | | 37° C. | | | |
| 30 mL | | RPMI + P | 4° C. | Made in | |
| | | | | lab | |
| One 2-mL | 0.05%/ | Trypsin/ | Hannah-20 | | |
| aliquot | 0.02% | EDTA | | | |
| EVOS RFP filter cube | | | | Life | |
| | | | | Sciences | |

Example Process

TABLE 30
(I) 20 minutes before start: Warm up reagents, new flasks and plate prior to beginning the
protocol. (A) Aliquot and place in bead bath: (i) 10 mL RPMI + P; (ii) 10 mL PBS; and (iii) 1
aliquot 0.05% Trypsin. (B) Prepare a T-75 with 20 mL RPMI + P and place in incubator.
(II) In BSC: Passage H441s:
(A) Aspirate old media from the T-75
(B) Rinse with 10 mL warm PBS and wait 3 min.
(C) Aspirate PBS
(D) Add 2 mL 0.05% trypsin-EDTA (1 aliquot) and swirl around the flask to cover cells
evenly.
(E) Observe cells on the DMI-1 until they are rounded and begin detaching.
(F) When they appear fully rounded, swirl the flask and tap on your palm to lift. Do not tap on
the bench, it's too rough on cells and may create clumps.
(G) When >95% of cells are lifted on DMI-1 inspection, return to the BSC.
(H) Tilt the flask to collect cells in the bottom corner. Use a 5 mL serological to pipette cells
up and down to break up clumps before trypsin inactivation.
(I) Using a 10 mL serological, add 5-10 mL RPMI + P to inactivate trypsin: (i) Tilt the flask and
pipette along the bottom of the flask to detach cells and flow them to the corner; (ii) Pipette up
and down several times to disrupt clumps; and (iii) Collect cells and place in 15 mL Falcon
tube.
(J) Centrifuge to pellet cells for 5 min at 200 g, 25 C. During the centrifuge, prepare for cell
counting and seeding: (i) Bring in P100 pipette tips; (ii) Bring in a 0.5 mL Eppendorf tube;
(iii) Turn on the cell counting computer & open the software; (iv) Peel the plastic off the
counting chamber; and (v) Bring the newly warmed up T-75s into the hood & label with
initials, cell type, passage number & date.
(K) In BSC: After centrifuge, aspirate the supernatant and resuspend cells. Pipette the top of
the cells to gently disrupt the pellet. Try not to lift up or aspirate the pellet.
(L) Add 2 more mL RPMI-P to dilute cells for accurate counting. Cell counter precision can
be greatly reduced at high cell concentration (>2e6 cells/mL)- personal observation.
(M) Count H441s several times by shifting the counting chamber's position. Choose the
lowest number as the cell density, and write this number down. Repeat for each flask.
(N) Seed the two fresh T-75's with 1e6 cells each and return to the incubator.
Notes:
The cells should be passaged at 70-90% confluence. If you want to get to the next passage sooner than 6 days, you can seed at 1.5e6 cells/flask instead of le6 and you can passage in 4 days. Don't dilute the cells too much during routine passaging (more than 1:10) as you can cause genetic drift of the cell population.

v) H441 Phenotype in T-75s.

If there isn't enough media, the cells may show a mesenteric phenotype with spindles, holes between cells and a squamous shape. With adequate media they display an epithelial phenotype with clumped “islands” and tight association between neighboring cells. If they become starved they can transition from the epithelial phenotype to a squamous one. They can usually be recovered if they are fed. Sometimes they are too far gone though, if you can't recover the good phenotype you should start over with a new batch of cells.

E) HUVEC-RP Maintenance in T-75 flasks.

i) Making Complete VCBM Media.

Materials

TABLE 31

| | Concen- | | | | Product |
|---|---|---|---|---|---|
| Volume | tration | Material | Notes | Vendor | Number |
| 1 | kit | Endothelial | −20° C. | ATCC | ATCC ® PCS- |
| | | cell growth | | | 100-041 ™ |
| | | kit-VEGF | | | |
| 1 | bottle | Vascular cell | 4° C. | ATCC | ATCC ® PCS- |
| | (475 mL) | basal medium | | | 100-030 ™ |
| 5 mL | 100× | penicillin/ | −80° C. | aliquoted | |
| | | streptomycin | | | |
| | | (pen-strep) | | | |

Example Process

TABLE 32
(1) Obtain one growth kit from the freezer.
(2) Thaw the components of the growth kit just prior to adding
them to the basal medium. Warm the L-glutamine component
in a 37° C. water bath and shake to dissolve any precipitates,
prior to adding to the basal medium. If precipitates
persist after shaking, add the L-glutamine anyway,
rinse all the precipitates into the bottle, and shake/
rock the media bottle until precipitates dissolve.
(3) Obtain one bottle of Vascular Cell Basal
Medium (475 mL) from cold storage.
(4) Transfer the volume of each growth kit
component to the bottle of basal medium:

| | | | Final | |
|---|---|---|---|---|
| Component | Volume | | Concentration | |
| rh VEGF | 0.5 | mL | 5 | ng/mL |
| rh EGF | 0.5 | mL | 5 | ng/mL |
| rh FGF basic | 0.5 | mL | 5 | ng/mL |
| rh IGF-1 | 0.5 | mL | 15 | ng/mL |
| L-glutamine | 25.0 | mL | 10 | mM |
| Heparin sulfate | 0.5 | mL | 0.75 | Units/mL |
| Hydrocortisone | 0.5 | mL | 1 | μg/mL |
| hemisuccinate | | | | |
| Fetal Bovine Serum | 10.0 | mL | 2% | |
| Ascorbic acid | 0.5 | mL | 50 | μg/mL |

(5) Add Penn-strep: 0.5 mL 100× (10 units/mL pen, 10 ug/mL strep)
(6) Tightly cap the bottle of complete growth medium and swirl the
contents gently to assure a homogeneous solution. (A) Do not shake
forcefully to avoid foaming. (B) Label and date the bottle.
(7) Complete media should be stored in the dark at 2° C. to 8° C.
(do not freeze). When stored munder these conditions,
complete media is stable for 30 days.

ii) Passaging HUVEC-RFP.

Materials

TABLE 33

| | Concen- | | | | Product |
|---|---|---|---|---|---|
| Volume | tration | Material | Location | Vendor | Number |
| 1 T-75 | 60-80% | HUVEC | 37° C. | ATCC | |
| | confluent | T-75 | incubator | | |
| 10 mL | 1× | PBS | 4° C. | | |
| 20 mL | | VCBM + | 4° C. | ATCC | |
| | | S | | | |
| 2 mL | 0.05% | Trypsin/ | −20° C. | Corning | |
| | | EDTA | | | |

Example Process

TABLE 34
(1) Warm up in bead bath: (A) 2-mL 0.05% trypsin aliquot; (B) 10-mL PBS aliquot in 37° C.
bead bath.
(2) Pipette 25 ml of VCBM to a new T-75 flask and warm in the incubator.
(3) From old flask: Aspirate out old media.
(4) Rinse with 15 mL pre-warmed PBS. Allow it to sit 3-5 min.
(5) Repeat previous step for second PBS rinse to completely remove media (it can neutralize
trypsin).
(6) Pipette 2 mL trypsin onto cells and gently rock flask to make sure it covers all cells.
(7) Wait 1-2 minutes for cells to become rounded- observe on inverted microscope brightfield
5×.
(8) When cells are rounded, gently shake and/or tap on the palm to detach if needed.
(9) Add 1 aliquot (5 mL) trypsin neutralizing solution to the flask.
(10) Transfer the cell suspension to a 15 mL conical vial.
(11) Centrifuge the cells to pellet- 5 min, 200 g, 25 C.
(12) While centrifuging, examine flask under microscope and if there are still >10% of cells
attached, repeat steps 5-9 if you think you'll need the rest of the cells to have enough.
Otherwise discard.
(13) Aspirate supernatant without disturbing the pellet.
(14) Resuspend the cells in 1 mL media or PBS.
(15) Count cells in cell counter.
(16) Seed 5k cells/cm\hat{ }2 for rapid growth or 2.5k cells/cm\hat{ }2 for regular subculture.
NOTE:
Passage at 60-80% confluence. DO NOT let HUVECs get overconfluent. If they pass 80% confluence, they won't be useable in Transwell coculture. They may die before Day 8. It is preferable to increase passage number rather than let HUVECs crowd in the flask.

iii) RNA Extraction from Epithelium and Endothelium Separately.

Materials

TABLE 35

| | | Concen- | | | | Product |
|---|---|---|---|---|---|---|
| Volume | | tration | Material | Location | Vendor | Number |
| 1 | plate | Epi-endo | | 37° C. | | |
| | | coculture | | incubator | | |
| 20 | mL | 0.25% | Trypsin- | −20° C. | Thermo- | 25200056 |
| | | | EDTA | | Fisher | |
| | | | | | Scientific | |
| 50 | mL | 1× | Phosphate- | 25° C. | Thermo- | 10010023 |
| | | | buffered | | Fisher | |
| | | | saline (PBS) | | Scientific | |
| 50 | mL | | Trypsin | −20° C. | Lonza | CC-5002 |
| | | | neutralizing | | | |
| | | | solution | | | |
| | | | (TNS) | | | |
| 1 | kit | | Takara Bio | PCR | Takara | 740698.5 |
| | | | NucleoSpin 8 | station | Biosciences | and |
| | | | or 96 RNA | | | 740684 |
| | | | extraction kit | | | Or |
| | | | plus | | | 740709.4 |
| | | | accessories | | | |
| | | | and reagents | | | |
| 1 | kit | | Agilent RNA | −20° C., | Agilent | 5067- |
| | | | 6000 | 4° C., | | 1513 |
| | | | Pico Kit | PCR | | |
| | | | | station | | |

Equipment

TABLE 36
Equipment

96-well rotor for tabletop centrifugation with 6000 g capability
Agilent 2100 Bioanalyzer System
V-well 96-well microplate

Example Process

TABLE 37
(1) Ahead of experiment day, prepare Takara Bio RNA kit and Agilent Bioanalyzer kit
reagents as described in manufacturers' instructions.
(2) Warm up in bead bath for 20 minutes: (A) Trypsin-EDTA; (B) Trypsin neutralizing
solution; and (C) PBS.
(3) Wash the cells prior to trypsinization: (A) Using a 12-well multichannel pipettor or a liquid
handler, remove the liquid from the top and bottom wells of the co-culture plate. Replace with
200 uL/well (bottom) and 150 uL/well (top) warmed PBS; (B) Repeat part a) to remove and
replace the PBS for a second wash step; and (C) Completely remove the PBS from the top and
bottom chambers.
(4) Trypsinize and collect cells: (A) Add 100 uL/well (bottom) and 50 uL/well (top) of pre-
warmed 0.25% trypsin-EDTA to the cells and incubate in a humid 37° C. cell culture incubator
for 15 minutes. NOTE: Trypsin should be completely warmed to 37° C. prior to this step. Cold
trypsin will have less activity and this will compromise trypsinization. (B) While incubating
the plate, prepare TNS in two separate 300-uL/well capacity V-well 96-well microplates by
adding 100 uL/well TNS to each well. Also prepare a reagent reservoir with the leftover 20
mL of TNS. (C) After trypsin incubation, use a multichannel pipette or liquid handler to
pipette up/down 15 times in the top chamber and then bottom chamber to detach cells from the
membrane. Then, collect as much liquid as possible from the top chamber into the first TNS
plate, and from the bottom chamber into the second TNS plate. Then rinse each well in the top
and bottom with 50 uL spare TNS to rinse extra cells off. Collect the 50 uL rinse into each
well of the V-well microplate. Make sure to pipette up/down multiple times per well to mix
the trypsin and TNS solutions. (D) Use the microplate-rotor centrifuge to spin down cells in
the V-well collection plate at 500 g for 5 minutes. (E) Carefully aspirate most of the liquid
from the wells without disturbing the cell pellet.
(5) Prepare Bioanalyzer kit: place the bioanalyzer 4° C. reagent box, along with one ladder
aliquot taken from −20° C., on the lab bench at 25° C. protected from light to adjust to room
temperature.
(6) Extract RNA: follow instructions from Takara Bio for the 96-well cell culture format.
(7) Quantify RNA concentration and integrity: Keeping eluted RNA on ice with lids on, take
samples and bioanalyzer kit plus chips immediately to the Agilent 2100 Bioanalyzer machine.
Prepare and run the samples as described in the manufacturers' instructions.
(8) As soon as possible, store and freeze eluted RNA at −80 C. prior to analysis.

Neutrophil Transmigration in 96-Well Co-Culture.

Materials

TABLE 38

| | | Concen- | | | | Product |
|---|---|---|---|---|---|---|
| Volume | | tration | Material | Notes | Vendor | Number |
| 1 | plate | Epi-endo | | 37° C. | | |
| | | coculture | | incubator | | |
| 50 | mL | 1× | HEPES- | 4° C. | Lonza | CC-5024 |
| | | | Buffered Saline | | | |
| | | | Solution | | | |
| 1 | kit | | MACSxpress ® | 4° C. | Miltenyi | 130-104- |
| | | | Whole Blood | | Biotec | 434 |
| | | | Neutrophil | | | |
| | | | Isolation Kit, | | | |
| | | | human | | | |
| 9-12 | mL | | Whole blood | 25° C. | | |
| | | | (human) | | | |
| | | | in EDTA | | | |
| 1 | | aliquot | Leukotriene | −20° C. | Sigma | L0517- |
| | | | B4 (LTB4)* | | Aldrich | 25UG |

*LTB4 should be aliquoted and stored in plastic under inert gas (argon or nitrogen) at −20 C. Do not thaw or open until use. Aliquots cannot be reused.

Equipment

TABLE 39
Equipment

| | |
|---|---|
| MACSxpress LRSC Starting Kit | 130-120-192 |
| V-well 96-well microplate | |

NOTE:
whole blood should be drawn immediately prior to neutrophil isolation, and neutrophils should be used immediately after isolation.

Example Process

TABLE 40
(1) Warm up the HEPES in 37° C. bath for at least 20 minutes.
(2) Prepare the chemoattractant plate with LTB4 as a control, or other samples as test wells.
Place samples in a 96-well HTS receiver plate. LTB4 aliquot should be suspended in HEPES
at 0.1 ug/mL. Place the chemoattractant plate in a 37° C. humid cell culture incubator to warm up.
(3) Acquire fresh human whole blood. Follow the manufacturer protocol for the MACSXpress
kit isolation of human neutrophils.
(4) Once neutrophils are isolated, wash the cell coculture plate twice with HEPES: (A) Remove
all media from the top and bottom chambers; and (B) Add HEPES to the plate (150 uL top and
200 uL bottom) and remove. Repeat.
(5) Count and resuspend neutrophils at 3.5 million cells/mL in HEPES buffer.
(6) Move the insert plate into the chemoattractant receiver plate. Immediately pipette 100
uL/well neutrophil suspension at 3.5e6 cells/mL into each well. Place the plate into a humid cell
culture incubator for at least two hours.
(7) Neutrophil chemotaxis can occur within two hours of neutrophil addition. However, assay
can be run overnight to yield high neutrophil count.

### Example 3. Generation of a Humid Environment for Consistent Moisture and Reduction of Edge Effects in 96-Well HTS

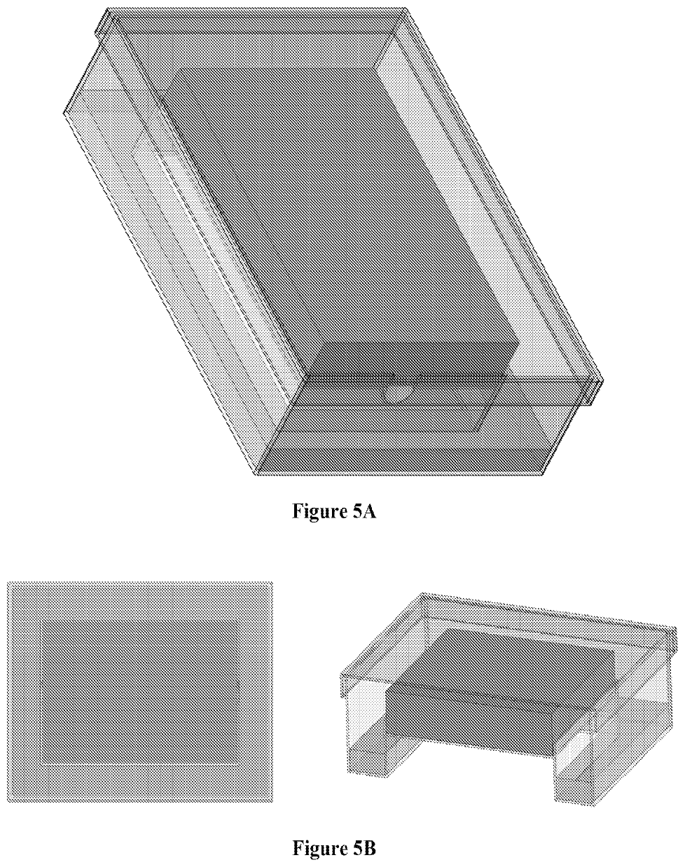

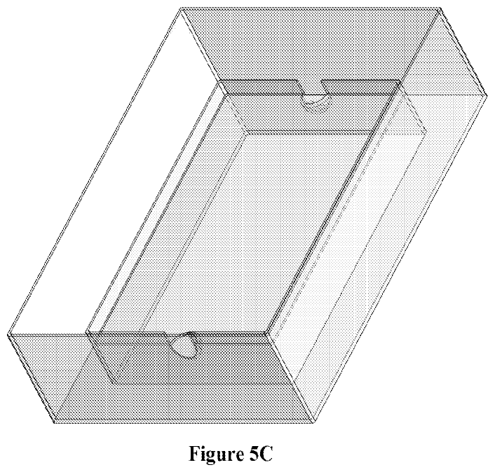

It is well established that uneven humidity in microwell plates can affect cell-based assays and that enhanced humidity of the immediate environment largely negates such edge effects. While custom microplates are available to create a plate-surrounding water moat that substantially reduces edge effects, the Corning Transwell HTS plate system cannot be used with such custom plates. Additionally, the close proximity of the humidifying liquid to samples presents a contamination risk. Therefore, a custom microplate-containing humidifier box was designed, described in FIG. 5.

### Example 4. In Vitro Assessment of Chemical Mixture-Induced Airway Inflammation in Healthy and Diseased Lungs

Evaluate and Promote the Use of Cell- and Tissue-Based Assays that More Accurately Represent Human Susceptibility than Animal Models to Adverse Reactions.

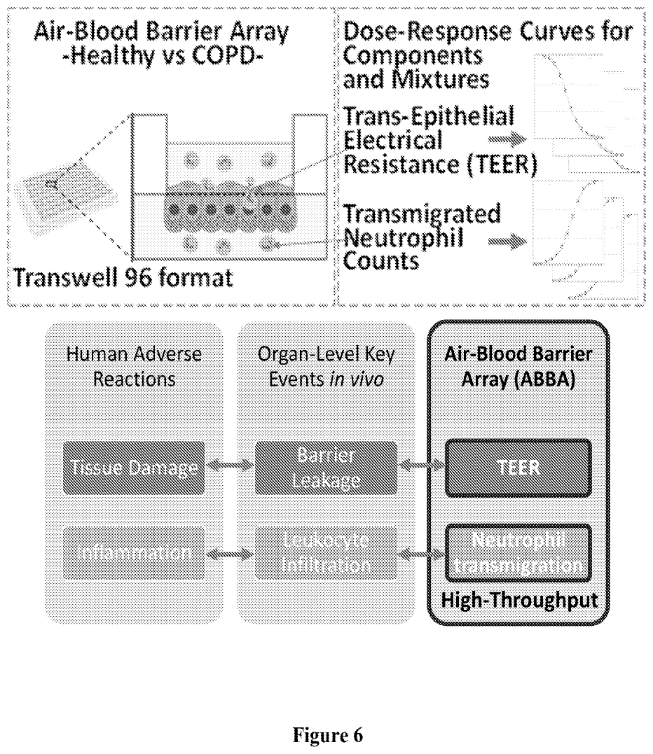

The exemplary system and method can be used to determine benchmark doses (BMD), e.g., for electronic nicotine delivery systems (ENDS) constituents and mixtures using organ-level adverse reaction readouts in vitro. Use in vitro to in vivo extrapolation (IVIVE) to correlate in vitro responses to in vivo human exposure risk. Immunophenotype. Organ-level key readouts can better predict human adverse reactions. High-throughput, continuous data readouts provide full dose responses for BMD. (FIG. 6). This method can be used to fill the rodent-human species gap, molecular level-organ level response gap (e.g., cytokine expression does not predict neutrophilia), and health-disease gap (COPD patients can be more prone to adverse reactions). The method can be used to enable analysis of joint toxicities of mixtures.

In some embodiments, the method includes (1) establishing a 96-well format, human cell, air-blood-barrier array (ABBA); (2) obtaining full dose-response curves for barrier leakiness and neutrophilia; and (3) analyzing molecular/cellular profile of healthy versus COPD airway milieu.

Discussion. Assessing adverse reaction risk of inhaled substances, including Electronic Nicotine Delivery Systems (ENDS) aerosols and other new and emerging tobacco products or their constituents, is important yet difficult using existing in vitro methods. Rodent studies are not only low-throughput and presenting with ethical issues but also have species-dependent toxicity pathway differences [e.g., aromatic hydrocarbon receptor (AHR) specificity and response] that limit prediction of adverse outcomes in humans.

The current gold standard is primary human lung epithelial cells cultured at an air-liquid interface (ALI). While very useful, the throughput and sensitivity of this method can be low. Due to lack of leukocytes and endothelial cells, organ-level adverse responses (e.g., neutrophilia) are difficult to predict. What can be beneficial is a high-throughput in vitro model that directly provides organ-level key event readouts such as neutrophil airway infiltration and air-blood barrier (ABB) deterioration.

In some embodiments, the exemplary method and associated system can be used to provide air-blood-barrier arrays (ABBAs) where the endothelium comprises the insert membrane upper-side and the ALI-cultured epithelium covers the membrane under-side. This geometry can allow primary human neutrophils added into the Transwell insert to settle onto the endothelium, then transmigrate from the ABB upper-side, through the endothelium and epithelium, to the under-side. All ABBA experiments can quantify trans-epithelial-electrical resistance (TEER), which can measure air-blood barrier function, and the number of transmigrated neutrophils. Detailed molecular and cellular analysis can also be subsequently performed for mechanistic analysis including: neutrophil phenotyping, bacteria phagocytosis assay, supernatant cytokine analysis, and epithelial and endothelial cells transcriptomics. For example, analysis of neutrophil phenotype change can help predict and/or assess risk for infective exacerbations in COPD patients that are attributed, not to lack of neutrophil numbers in the airways, but to their dysfunctional state.

As a clinically-relevant test, the ABBA-based in vitro neutrophilic airway inflammation response can be analyzed using airway fluid supernatant (ASN) prepared from COPD patient sputum. The project collects sputum from COPD patients that: (i) have stopped smoking, (ii) still smoke cigarettes, (iii) use both cigarettes and ENDS. ABBA response to these ASN can serve as physiologic references for COPD and cigarette/ENDS use.

These experiments can align with the interest area of FDA regulatory science to modernize toxicology to enhance product safety, which can improve the toxicologic and pharmacologic tools used to minimize risk and evaluate product safety and efficacy by conducting internal and collaborative research and development.

In some embodiments, the exemplary method can be used to provide a high-throughput in vitro air-blood barrier array (ABBA) based barrier breakdown and neutrophil transmigration/activation assay to predict human adverse reactions to inhaled substances. Dose response curves and benchmark doses (BMD) can be determined for a range of well-known and unknown substances including Electronic Nicotine Delivery Systems (ENDS) constituents. The organ-level functional readouts (TEER & transmigration) along with the ability to recover cells (neutrophils, endothelial cells, epithelial cells) and fluids from both airway- and blood-side fluids for molecular analysis can promote a better understanding of toxicity mechanisms across multiple levels of biological organization. In some embodiments, the exemplary method can also incorporates COPD patient-derived airway epithelial cells into the ABBA and expose the epithelial cells to COPD patient sputum-derived airway fluid supernatant (ASN) to recreate airway inflammation of COPD patients. In doing so, COPD patients can utilize ENDS as part of a smoking cessation program making interaction of COPD and ENDS substances relevant. The experiments are as follows (also see schematic in FIG. 10 below):

### Example Experiment #1. Air-Blood-Barrier Array (ABBA) Manufacture and Validation

The exemplary method can be used to establish the ABBA using normal and COPD primary human small airway epithelial cells. The method can characterize donor-to-donor variability including normal vs COPD, young vs. old, and male vs. female. The method can be used to develop calibration and normalization protocols based on TEER and transmigrated neutrophil counts using synthetic COPD airway fluid supernatant.

Background The lung's air-blood barrier is comprised of an epithelium, interstitium, and endothelium that together form a critical first line of defense against toxins and act as a regulator of access to the bloodstream. Despite this, the lung barrier is frequently modeled in vitro with only the epithelium especially for air-liquid interface (ALI) cultures. While epithelial tight junctions provide strong permeability restriction in the lung, the underlying endothelium can potentiate or compromise epithelial barrier function by paracrine signaling. In fact, loss of epithelial barrier function can originate from endothelial activation and dysfunction rather than direct epithelial insult.

Physiologically-relevant juxtaposed epithelial-endothelial co-cultures require the two cell types to be on opposite sides of a culture membrane. Typically, such co-culture is performed only in lower-throughput culture inserts that are seeded by manual inversion of the inserts. Such procedures are tedious, difficult to automate, and prone to failure particularly for the small 96-well inserts. A method was developed to eliminate the need for inversion by exploiting density-driven cell floating for underside cell seeding (FIG. 11). This project utilizes this method, but with the epithelial cells switched to primary cells and the endothelial cells switched from umbilical vein to pulmonary microvascular cells, to prepare the ABBA.

ABBA preparation: Briefly, polycarbonate 96-well HTS Transwell inserts with pore size 3 μm (Corning CLS3386) are collagen coated (30 μg/mL, 30 μL) then allowed to dry overnight. Primary human small airway epithelial cells (1.18e6 cells/mL) suspended in 50% Small Airway Epithelial Cell Growth Basal Medium (SAGM) (Lonza) and 50 vol % OptiPrep™ density gradient medium (STEMCELL Technologies 07820) are transferred to a 96-well receiver plate (Corning CLS3382) using a multichannel pipettor (85 μL/well). The collagen-coated inserts can then be placed into this plate. After 2 hours, 150 μL cell culture media can be added to the bottom, receiver plate chamber and 75 μL is added to the top chamber then allowed to incubate overnight. The next day, human lung microvascular endothelial cells (HMVEC-L, Lonza) (10,000 cells/well in 100 μL cell culture media) can be seeded into each top. Transwell insert chamber can be allowed to incubate overnight. These cultures can be transitioned to an ALI 48 hours after HMVEC-L seeding. To culture at ALI, the media can be removed from the bottom chamber and the media in the top chamber can then be replaced with serum-free 50/50 vol % epithelial and HMVEC-L cell culture media. Instead of serum, which can interfere with inflammatory responses, the media can contain, e.g., 1:50 v/v Ultroser G serum substitute (final concentration 0.2 mg/mL) (Sartorius 15950-017) to promote epithelial differentiation and polarization. The plate is cultured at ALI for 7-21 days.

ABBA calibration protocols: To validate, calibrate, and normalize the ABBA readouts for robust comparison of data across different plates, batches, and cell donors, the exemplary system can employ a defined-composition synthetic COPD ASN (sASN, e.g., that can compare dose-response curves (TEER and neutrophil count) with ABBA developed using cells from different donors towards this sASN.

The readouts can include barrier property breakdown (e.g., as measured by TEER) and neutrophil transmigration counts. Both of these readouts can provide continuous, as opposed to quantal, data and is obtained from each microwell. The results of dose response curves for TEER can change in response to different concentrations of extracellular DNA or histone and neutrophil transmigration to the chemoattractant IL-8 (18 hour transmigration time), e.g., as shown in FIG. 12.

Synthetic COPD airway fluid supernatant (sASN): A simple, defined mixture of a select chemoattractant (to promote neutrophil transmigration) and inflammatory biomaterial (to induce barrier breakdown) that are relatively stable and relevant to COPD sputum is formulated for use in calibrating each Transwell-96 ABBA. In the results (FIG. 8), a mixture of IL-8 (a relatively stable cytokine) and histone or NET-mimetic chromatin mesh suspension coined “microwebs” were tested.

Consideration of biological variables: The endothelial cell lot can be kept consistent throughout all studies by identifying and reserving vials of a suitable donor (from Lonza). Fresh neutrophils can be obtained from at least 4 different donors for each test. Epithelial cells from at least 4 different donors, 2 male and 2 female are tested. Lonza had normal primary epithelial cells from over 25 different donors in fall 2020, although the inventory is much lower recently. Epithelial cell donor is selected for sex and lots with large number of inventory (the number of available vials of cells from a single donor can vary from 1 to over 100 vials) to allow for repeated use of same donor cells for consistency. Dose-response curves with the sASN developed in the previous section are obtained using 4 different neutrophil donors, for the 4 different types of normal lung ABBAs prepared with epithelial cells from the 4 different donors.

### Example Experiment #2. Determination of Dose Response Curves and Thresholds

In some embodiments, the exemplary method can be used to determine the full dose-response curves for TEER and neutrophil counts for well-known toxins and for less-characterized ENDS constituents. Determine benchmark doses (BMDs) for individual constituents and mixtures. Perform IVIVE to determine human risk.

In some embodiments, the exemplary is sued to evaluate cigarette smoke extract (CSE), ENDS vapor extract (EVE), Nicotine, benzo[a]pyrene [B[a]P (a well-known carcinogen and arylhydrocarbon receptor (AhR) ligand]), and ENDS constituents. The exemplary method can also be used to evaluate mixtures such as B[a]P and EVE or COPD ASN are also tested.

Benchmark dose (BMD): Once dose response curves are obtained, benchmark dose analysis can be conducted, e.g., following the EPA Benchmark Dose Guidance [EPA 2012]. BMD and BMDL (lower confidence limit) can be calculated for the observed dose-response data for each substance and mixture. BMD values are calculated using EPA's Benchmark Dose Software (available from the EPA). The data can be modeled as continuous data using polynomial models, power models, and Hill models. The benchmark response can be set to 10% meaning 10% decrease relative to control for TEER response and 10% of maximal infiltration cell number (induced using high concentration of sASN) for neutrophil infiltration response. Models with a goodness-of-fit P<0.1 are excluded. The best model can be selected based on the lowest Akaike's Information Criterion (AIC) value.

Evaluating joint toxicity of mixtures: Analysis can be performed to investigate if the combined effect in the mixture leads to additive, synergistic, or antagonistic interactions. For each biological endpoint, three different methodologies are used to assess the mixture effect: concentration addition (CA), independent action (IA), and general concentration addition (GCA).

### Example 5. Procedure for Underside Air Liquid Interface (ALI) Cell Transmigration Assay

Visual barrier property confirmation: Underside ALI culture beneficially facilitate the visual identification of leaky barriers. That is, if any liquid has leaked from inside the Transwell insert into the bottom receiver well, then that indicates that the barrier property was bad and fluid leaked through the air-blood barrier. The underside ALI can also allow for convenient barrier property quality control. No leaking is in and of itself a quality control measure of barrier fidelity. The lack of such leakage is generally an indication that TEER is at least 100 ohm*cm².

Histological barrier property confirmation: Although this exemplary method may employ fixing and staining of the staining (whereas TEER is a non-terminal assay) histology does provide molecular confirmation of barrier properties. Using conventional immunohistochemistry, barrier properties can be confirmed.

Exposure to drugs, toxins, cytokines, other molecules: In addition, molecules to be tested can be added to just the upper side of the Transwell insert, the underside received plate well only, or both. When exposure includes the underside ALI, the air-blood barrier can conveniently be reverted to ALI culture by lifting the entire Transwell insert array from a liquid filled receiver plate to an empty receiver plate with minimal or no liquid such that the underside is air exposed.

Calibration with a known mixture: The air-blood barrier array can beneficially provide sufficient throughput and reproducibility to obtain dose response curves. While there may be plate-to-plate or day-to-day variability in exact quantitative response of the air-blood barrier and the cell transmigration rate, the exemplary method may include performing a calibration using a biomolecular mixture known to stimulate a combination of pathways in a way the mimics aspects of diseased airway fluid. One such calibration mixture may use include a mixture of lamnda phase DNA (methylated or unmethylated), histone (e.g., from calf histone), and human IL-8.

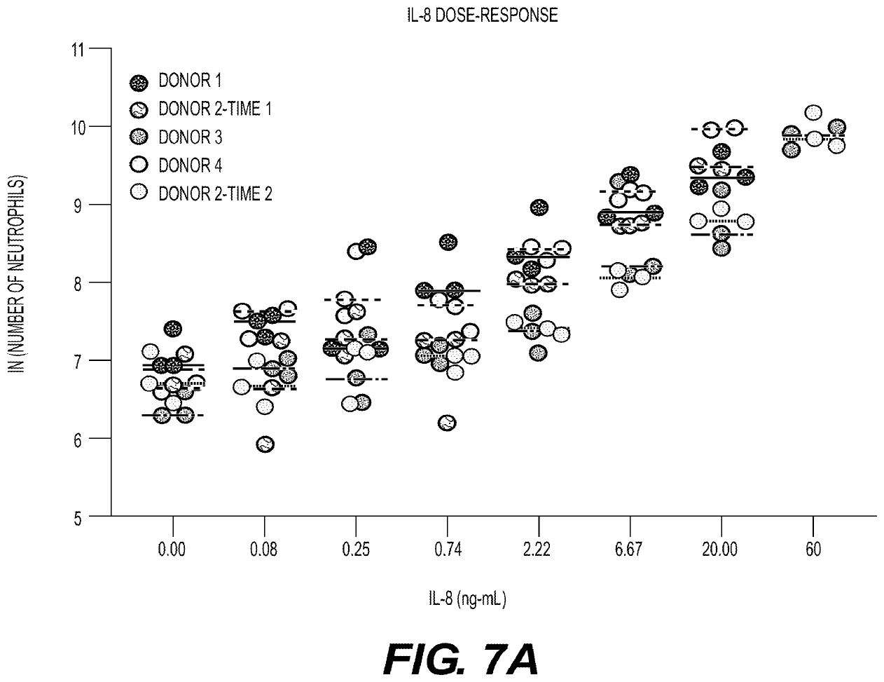

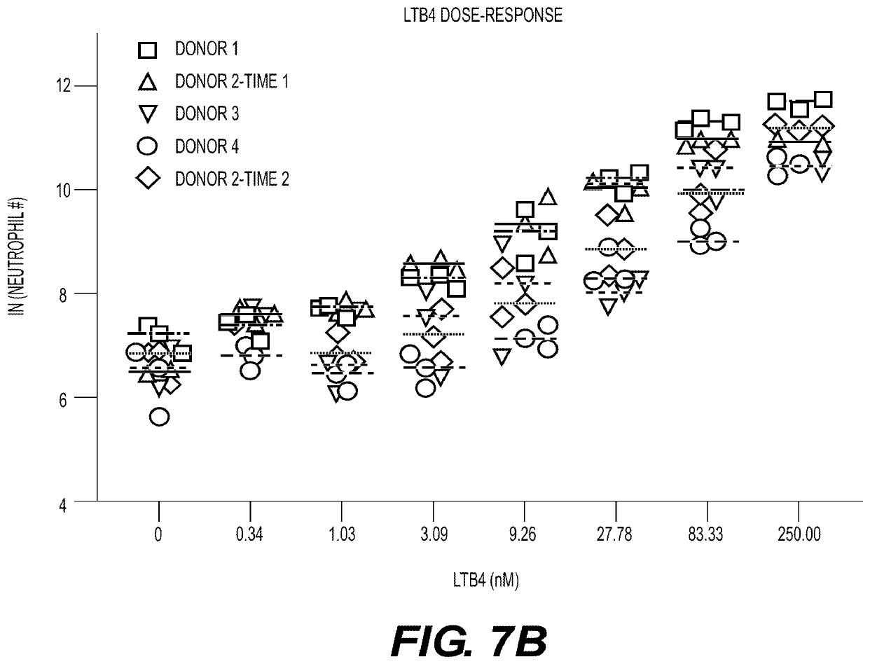

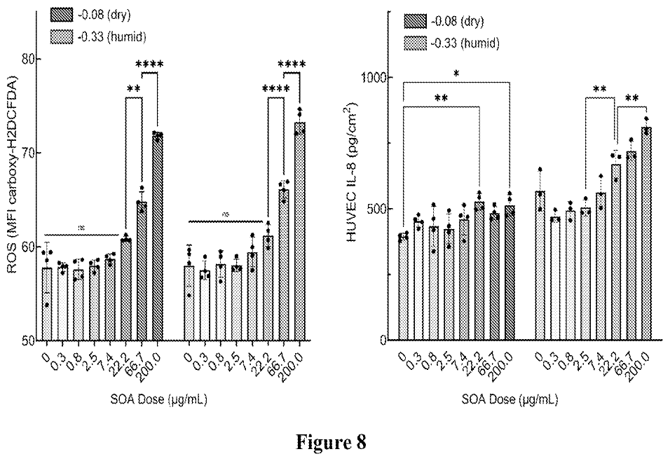

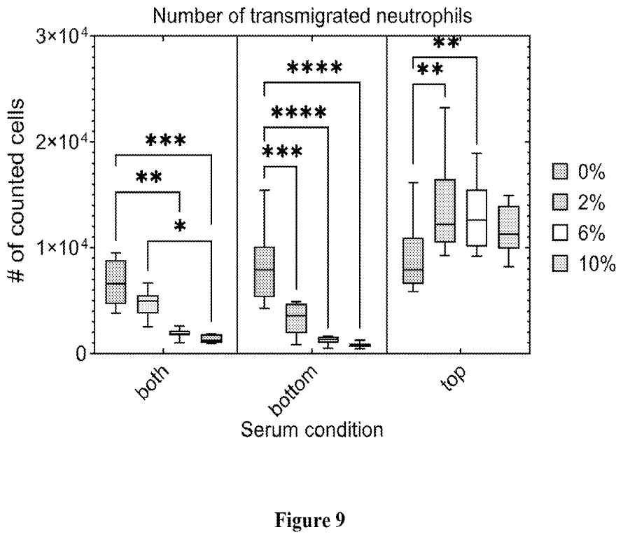

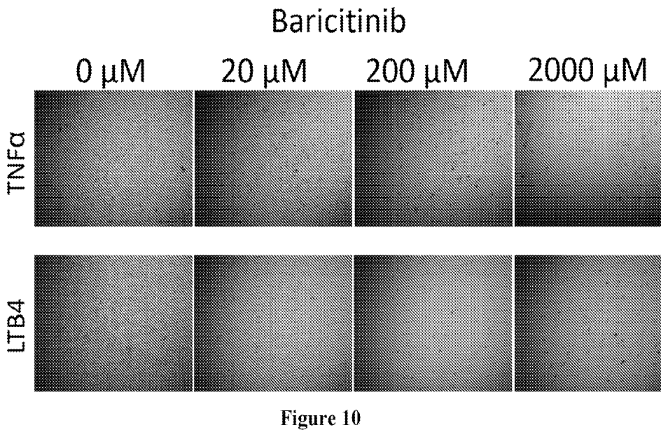

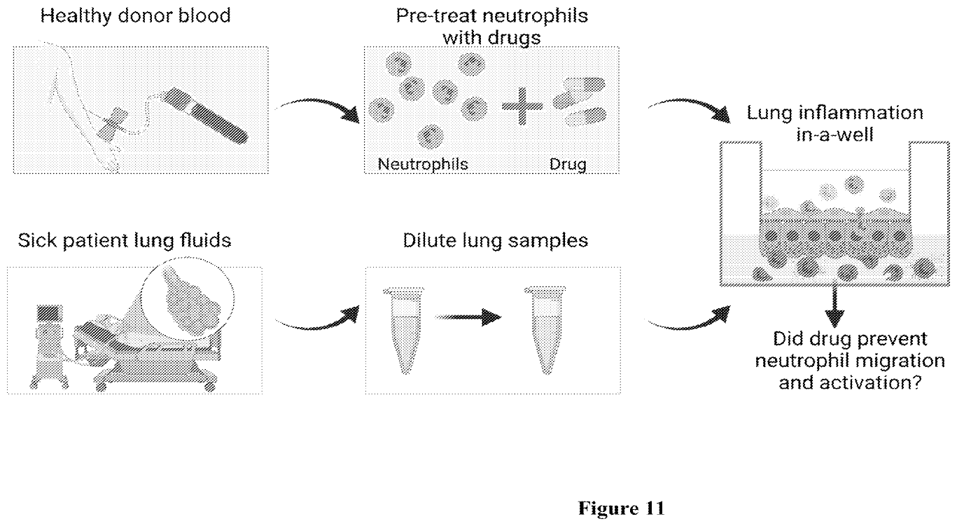

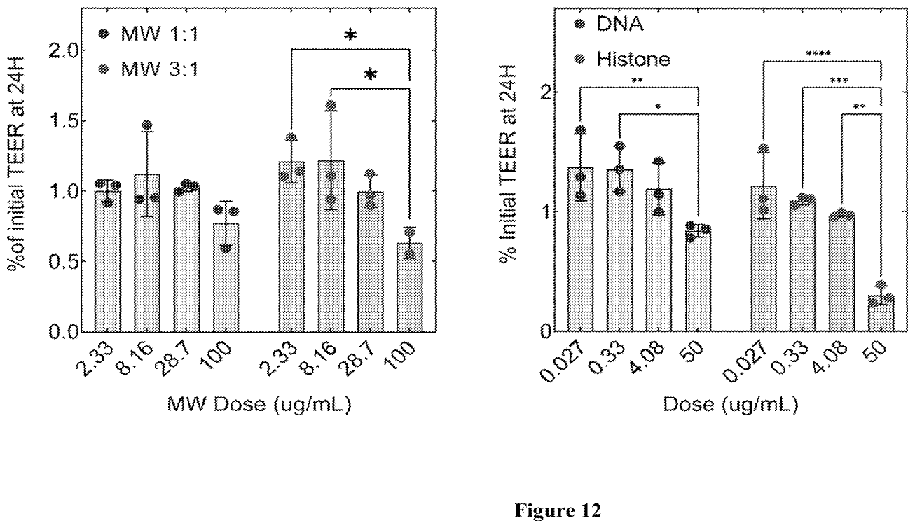

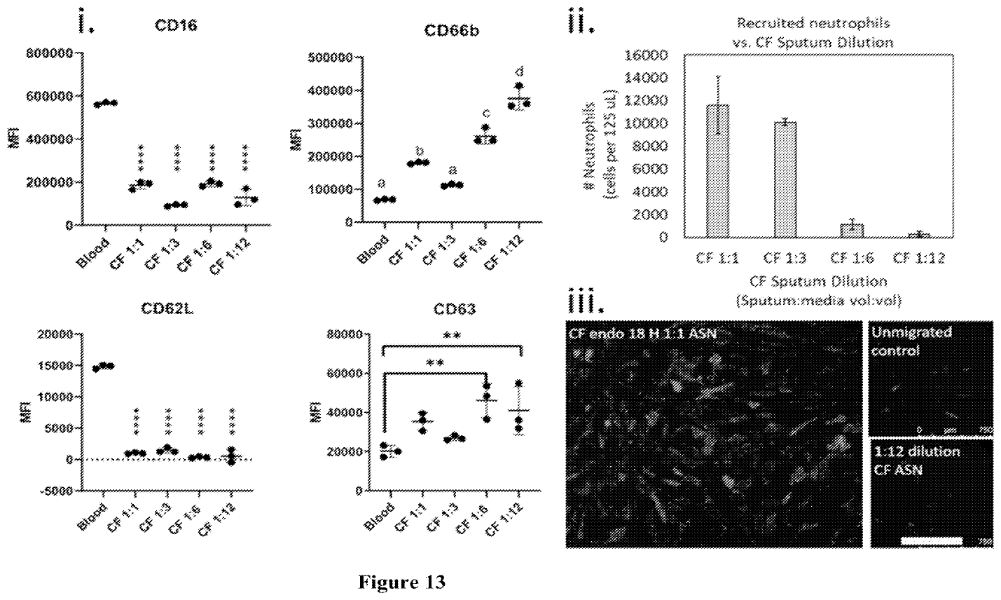

Example results of dose response curves for TEER change in response to different concentrations of extracellular DNA or histone and neutrophil transmigration to the chemoattractant IL-8 (18 hour transmigration time) are shown in FIG. 7.

Dose-response curves. Certain full dose response curve may employ 16 Transwell microwells within a plate. TEER decreases with increasing concentrations of DNA or histone. Neutrophil transmigration numbers increase with increasing IL-8 concentrations.

A simple, defined mixture of a select chemoattractant (to promote neutrophil transmigration) and inflammatory biomaterial (to induce barrier breakdown) that are relatively stable and relevant to diseased airway fluid can be formulated for use in calibrating each Transwell-96 ABBA. A mixture of IL-8 (a relatively stable cytokine) and histone or NET-mimetic chromatin mesh suspension coined “microwebs” a useful mixture (see FIG. 8).

Drug testing: In some drug testing applications, rather than treating the endothelial cells or epithelial cells, the exemplary method can pretreat the transmigrating cells (such as neutrophils) as well. In an example procedure, neutrophil suspension can be treated with different concentrations of a drug (baricitinib), then their ability to transmigrate towards chemoattractant can be monitored (FIG. 10). Primary peripheral human neutrophils were isolated from fresh whole blood and residual erythrocytes were depleted according to manufacturer instructions (Miltenyi Biotec #130-104-434, MACSxpress® Whole Blood Neutrophil Isolation Kit, human; Miltenyi Biotec #130-098-196 MACSxpress® Erythrocyte Depletion Kit, human). The untouched neutrophils were centrifuged at 200 g for 5 minutes to remove supernatant. The neutrophils were resuspended in 2 mL ALI media and counted on the T4 Nexcelom Cell Counter, ensuring that cells and borders were properly identified. Neutrophils were further diluted to 3 million cells/mL in ALI media. Baricitinib (Cayman Chemical #16707) was diluted into the neutrophil suspension at 2, 20, or 200 μM in ALI media. Neutrophils in ALI media with Baricitinib were incubated at 37 C for 2 hours. A 96-well Transwell coculture plate was prepared ahead of the experiment and was differentiated at ALI for 5-7 days on the day of this experiment. Immediately prior to placing neutrophils in the Transwell coculture plate, chemoattractants were placed in the bottom chamber of the Transwell plate. Specifically, chemoattractant compound TNF-α (170 ng/mL) or LTB4 (300 nM) was added to ALI media. 85 μL of either TNF-α or LTB4-containing media was placed in the bottom chamber of the Transwell in contact with the epithelium. To initiate the transmigration assay, neutrophil suspensions containing Baricitinib, that had been incubated for 2 hours prior, were placed directly into the top compartment of the Transwell in contact with the endothelium. The entire apparatus was incubated a further 16 hours at 370 C, 5% C02, 95% relative humidity. After this time, the number of migrated neutrophils was assessed by brightfield microscopy. The number of migrated neutrophils was qualitatively assessed to be reduced in the presence of increasing concentration of Baricitinib.

Flow cytometry: Despite the smaller wells and fewer number of cells involved, the 96 Transwells still provide sufficient cell number for flow cytometric analysis. In some embodiments, day 5-6 plates may be used for exposure (if overnight), and day 6-7 plates may be used for transmigration. The results may be maintained in spreadsheet (e.g. flowjo, plate reader, graphpad etc.). Migrated cells (in this example, neutrophils) can be analyzed by flow cytometry following cell migration, such as the exemplary results shown in Figures X, Y, and Z. An exemplary method of cell isolation, staining, and counting to produce such results is provided herein. Note that for all of the following procedures, neutrophils are handled on ice and with 4° C. reagents wherever possible. This is imperative to avoid unintentional activation and subsequent aggregation of the neutrophils that renders analysis impossible.

Cell collection. Following transmigration, the Transwell insert tray is moved to an empty receiver plate. The receiver plate containing migrated cells (hereafter the “receiver plate”) is then prepared for cell collection. The plate is placed on the bench, and oriented so that column 1 of the plate is oriented parallel to the scientist. Well A1 is oriented in the bottom left corner. The receiver plate is tilted by propping the long edge (parallel to row A) atop the receiver plate's lid. This helps collect the small media volume into one corner of each well. Cold PBS-EDTA is prepared. Then, using a 12-channel pipettor, media is collected from each well into a 96-well non-tissue culture treated round-bottom plate (Corning #3788) placed on ice (hereafter the “collection plate”). After each row is collected, fresh tips are gathered, and the row receives 100 μL/well ice-cold PBS-EDTA. After all 8 rows are collected into the round bottom plate, the process is repeated. That is, the 100 μL/well PBS-EDTA in the receiver plate is moved into the analogous row of the collection plate, and replaced with 100 μL/well fresh PBS-EDTA. Finally, after all 8 rows, the final collection is performed. The remaining 100 μL/well PBS-EDTA in the receiver plate is moved to the analogous row of the collection plate, ensuring that all volume is removed, even if some air is aspirated. In total, the collection plate ends the process with 285 μL/well, comprising 85 μL of medium from the transmigration assay, and 200 μL PBS-EDTA for washing the neutrophils out of this medium. To isolate neutrophils, the plate is centrifuged in a tabletop centrifuge at 400 g for 5 minutes at room temperature (Sorvall ST 16 Tabletop Centrifuge with M-20 Microplate Swinging Bucket Rotor). Counterbalance plate is prepared with equal media volumes, and verified with a microscale prior to centrifugation. Following centrifugation, the collection plate is placed on ice, again with A1 in the bottom left and column 1 parallel to the scientist. The supernatant is carefully aspirated using a 12-channel micropipette with fresh tips for each row. To ensure that the small cell pellets were not disturbed or aspirated, the tips are oriented at an approximately 30 degree angle to the plate surface to avoid contacting the bottom of the well (where the pellet is located) with the tips. Aspiration is performed slowly to avoid disturbing pellets. Before and after supernatant aspiration, pellets are visually inspected by holding the plate against fluorescent lights (i.e. ceiling lights) to visualize the cell pellets. If cell pellets are significantly disturbed or disappeared following supernatant aspiration, the well is eliminated from data analysis due to experimental error. The author notes that supernatant collection using automated liquid handling equipment (e.g. Integra Biosciences Viaflow 96/384) is possible, but inadvisable, because supernatant collection speed is too fast to avoid disturbing pellets.

Staining cells for flow cytometry without antibody staining. Migrated cells can be quantified on flow cytometry with or without staining for surface or intracellular markers. However, in all cases, viability is quantified. To count without antibody staining, the following procedure is performed. Following cell collection, pellets are resuspended into live/dead labeling solution (Biolegend Zombie NIR™ Fixable Viability Kit). To prepare the labeling solution, dye stock is prepared according to manufacturer instructions and diluted 1:500 into ice-cold PBS-EDTA. Cell pellets are resuspended into 100 μL/well labeling solution by aggressive pipetting using a multichannel pipettor. Cells are incubated for 10 minutes on ice in the dark. Then 200 μL/well ice-cold PBS-EDTA is added to each well to dilute the labeling solution, the plate is centrifuged at 400 g for 5 minutes, and supernatant is carefully removed as previously described for cell collection.

Staining cells for flow cytometry with antibody staining. To count with addition of antibody staining, the following procedure is performed. Following cell collection, pellets are resuspended into pre-stain solution containing live/dead labeling and Fc-blocking solutions (Biolegend Zombie NIR™ Fixable Viability Kit #423105; Biolegend Human TruStain FcX™ (Fc Receptor Blocking Solution) #422301). To prepare the pre-stain solution, dye stock is prepared according to manufacturer instructions and diluted 1:500 into ice-cold PBS-EDTA. Into the same solution, Fc-block is diluted 1:100. Cell pellets are resuspended into 100 μL/well pre-stain solution by aggressive pipetting using a multichannel pipettor. Cells are incubated for 10 minutes on ice in the dark. Then 200 μL/well ice-cold PBS-EDTA is added to each well to dilute the pre-stain solution, the plate is centrifuged at 400 g for 5 minutes, and supernatant is carefully removed as previously described for cell collection. The cells are then stained with antibodies targeting CD66b, CD63, CD16, CD62L, and Ep-CAM (Biolegend #392916, 353026, 302008, 304824, 118216, respectively) as follows. The master mix containing all antibodies is prepared by mixing 3 μL/well of each antibody and diluting to 100 μL/well with PBS-EDTA. For example, to prepare master mix for one 96-well plate, 100 wells' worth of solution is prepared by mixing 3*100=300 μL per antibody (total 1500 μL of antibody solution). Then, since each well requires 100 μL of solution, the total volume needed is 10 mL, so that 10-1.5=8.5 mL PBS-EDTA is added to the antibody mixture. Then each cell pellet is resuspended with 100 μL of this solution so that the well receives 3 μL of each antibody diluted to 100 μL with PBS-EDTA. The cells are incubated with antibody solution for 30 minutes on ice in the dark. The antibody solution is then diluted with 200 μL/well ice-cold PBS-EDTA, the plate is centrifuged, and supernatant is removed according to the previous description under “Cell collection.”

Cell fixation. For both antibody-labeled and unlabeled cells, fixation allows flexibility between the time of cell collection and time of flow cytometry. The previously listed antibodies are confirmed to be compatible with formaldehyde fixation, but other antibodies and combinations may require verification prior to experiments. Cells are fixed by resuspending the pellet in fixative after live/dead for unlabeled cells and after antibody staining for labeled cells. The cells are resuspended in 100 μL 2% paraformaldehyde (PFA) in PBS-EDTA and incubated for 5 minutes in the dark on ice. PFA is prepared by diluting 4× into PBS-EDTA from 16% PFA (Pierce™ #28906, 16% Formaldehyde (w/v), Methanol-free). Following incubation, wells are diluted with 200 μL/well ice-cold PBS-EDTA, the plate is centrifuged, and supernatant is removed according to the previous description under “Cell collection.” Finally, the pellets are resuspended in 75 μL/well ice-cold PBS-EDTA using a 12-channel pipettor with aggressive pipetting. At this stage, cells can be stored for up to 3 days prior to flow cytometry for antibody labeled cells, and for 7 days for non-labeled cells, provided that the plate is protected with a plate sealer to prevent evaporation and is stored in the dark at 4 C.

Flow cytometry for counting only. Cell counting in high throughput is challenging using traditional methods that require manual sampling, such as hemocytometry and automated benchtop cell counters like the Nexcelom T4 Cell Counter. Therefore, cell counting can be reliably performed on certain flow cytometers that report the number of events and the volume of sample collected with high accuracy and precision. In our application, the Cytoflex S (Beckman Coulter) is preferred for its accurate cell counting and automated microplate sampling system. Cells were counted on the Cytoflex S at 30 μL/min with a sample volume of 10 μL/well. This sampling volume is adequate to extrapolate the total cell number in the well, provided that the well is adequately mixed. Mixing is performed immediately before flow cytometry using manual pipetting followed by gentle vortexing. Mixing is also performed by the Cytoflex during sampling. Viability is determined by gating using a dead-cell reference. Specifically, a sample of 50-100 μL of live neutrophils sampled from the top compartment of one or two wells from the experiment is placed in a 65° C. oven for 5 minutes to induce cell death. The sample is then stained with Zombie NIR Fixable Viability Kit as described. This sample is run as a positive control on the Cytoflex S. Since only one color is measured, compensation is not necessary. After collecting this control and all samples, the data are uploaded to FlowJo™. The positive control cells are gated on FSC:SSC to identify the cell population, and then on SSC vs. APC-A750 to gate dead cells. This gate is then applied to the experimental cells. Only live cells are included in further analyses.

Flow cytometry for counting and antibody staining. For fold-change analysis, neutrophils isolated from whole blood are stained on the same day that transmigration is initiated, and this group is compared to transmigrated neutrophils that are collected and stained after 16 hours in the assay. A second control stain can be performed wherein unmigrated neutrophils from the top compartment of the Transwell can be collected at the same time as the migrated neutrophils for comparison of migrated vs. non-migrated cells that were both incubated in the Transwell assay for the 16 hour experiment duration. This control is considered less reflective of the neutrophil phenotypic changes induced by the assay than the fresh-blood control, and therefore the fresh neutrophil control is used to calculate fold-change in the data shown herein. Compensation is performed using compensation beads (Invitrogen™ #A10497 AbC™ Total Antibody Compensation Bead Kit). The beads are stained according to manufacturer instructions and collected under the same acquisition settings as the stained cells. Compensation is completed in FlowJo™ during analysis. Gating: Cells are first gated on FSC:SSC to remove doublets. This group is then gated by viability using SSC:APC-A750 for Zombie NIR viability stain. The same gates are applied to controls and experimental conditions. Only live cells are included in any analysis.

Statistics: Flow cytometric data is processed using FlowJo™ to produce raw data. This includes the number of live cells measured and the mean fluorescence intensity (MFI) of each marker measured after compensation and gating. This raw data is exported the GraphPad Prism 9 for statistical analysis. For analysis of multiple groups with one independent variable (e.g. dose of chemoattractant), one-way ANOVA is performed with post-hoc Tukey's t-test. For analysis of groups with two independent variables (e.g. dose of chemoattractant, identity of chemoattractant), two-way ANOVA with post-hoc Tukey's t-test is performed. In both cases, alpha=0.05 and t-tests are two-tailed.

Example parameters for experiment conditions are provided in Table 41.

TABLE 41

| | |
|---|---|
| Number of | |
| plates | |
| (e.g., 1, 2, 3) | |
| Example | i. Epi, endo or both |
| conditions | ii. length of exposure time |
| | iii. pre-exposure prior to TM. Or exposure during TM |
| Exposure | Consider outcomes of interest- e.g., timescale of |
| condition(s) | interest. For example, ROS is within first 30 min-2 hrs; |
| | cytokines within 24-72 hours |
| | Removal of pre-incubation media or not |
| | before transmigration. |
| | Direct neutrophils interaction with the stimulus. |
| | Or only interactiion with stimulated cells. |
| Number of | |
| donors | |
| Number | |
| of replicates | |
| per condition | |
| Number of | If Drug testing: what drugs, what doses. |
| controls, | |
| and control | |
| conditions | |
| Decide | Supernatant: ELISA/mesoscale (epi/endo what targets). |
| Outcome | Epi/endo: (e.g., (i) Live/dead (epi/endo/both/neither); |
| measures | (ii) permeability; (iii) TEER; (iv) immunostaining |
| | (epi/endo); (v) PCR (epi and endo can be done |
| | separately); (vi) ROS/live cell imaging (incucyte)). |
| | Neutrophils: (e.g., number of neutrophils, neutrophil |
| | flow panel (abs), functional assays (e.g., Seahorse, |
| | phagocytosis, bacterial killing, RNA (SYTO), ROS, |
| | etc.) |
| Make | Bottom map for exposure |
| plate | Top map for neutrophil donors/exposure conditions on |
| maps | top if needed |
| | Day 1 and day 2 maps if needed |
| | Label doses, controls, exposure conitions on the map |
| Calculate | (A) Perform ALL calculations ahead of experiment. If |
| dilutions, | cell count is needed, use a “dummy” number until the |
| doses, etc. | experiment. |
| required | (B) Calculate amount needed plus, e.g., 250 uL extra to |
| | account for volume losses. For example, vol needed for |
| | 10 wells is 850 uL, so make 1100 uL |
| | (C) Minimize waste of expensive reagents |
| Execute | |
| experiment | |
| Collect/ | (A) Take TEER after experiment |
| Process Data | (B) Measure neutrophil data on flow cytometer |
| | (C) Process flow data on Flowjo, image data on |
| | Incucyte software (if used incucyte) or ImageJ if used |
| | Evos for fluorescence |
| | (D) Use GraphPad Prism 9 for statistical analysis and |
| | data visualization |
| Day −1: Prepare | Measure TEER; |
| exposure | Apply exposure (if applicable); and/or |
| (if applicable) | Incubate overnight |
| Day 0: | Measure TEER (unless you measured yesterday for a |
| Transmigration | 24-hr exposure); |
| | Get blood draw; |
| | Separate neutrophils according to manufacturer |
| | instructions; and/or |
| | While neutrophils are separating: prepare bottom plate |
| | for transmigration. |

Once neutrophils are isolated, resuspend in ALI media at 3e6/mL, remove 100 uL/well from top well of plate, add 100 uL neutrophils at 3e6 cells/mL in ALI media to top well, place inserts in new bottom plate containing chemoattractant (or whatever your experiment calls for), incubate 2-16 hrs, determine if TM is working after about 30 min, include an LTB4 control condition on every plate (at least 3 wells of 100 nM LTB4), save and stain pre-TM isolated neutrophils if staining for flow cytometry analysis.

While the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims), including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure, including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

The following patents, applications and publications as listed below and throughout this document are hereby incorporated by reference in their entirety herein.

### REFERENCES

[1]. Larson, B., Banks, P., Sherman, H. & Rothenberg, M. Automation of Cell-Based Drug Absorption Assays in 96-Well Format Using Permeable Support Systems. J. Lab. Autom. 17, 222-232 (2012).
[2]. Huang, S. et al. Establishment and characterization of an in vitro human small airway model (SmallAir™). Eur. J. Pharm. Biopharm. 118, 68-72 (2017).
[3]. Gard, A. L. et al. High-Throughput Human Primary Cell-Based Airway Model for Evaluating Influenza, Coronavirus, or other Respiratory Viruses in vitro. bioRxiv 2020.05.23.112797 (2020) doi:10.1101/2020.05.23.112797.
[4]. Gobel, J. et al. A Phenotypic High-Throughput Screen with RSV-Infected Primary Human Small Airway Epithelial Cells (SAECs). J. Biomol. Screen. 20, 729-738 (2015).
[5]. Bluhmki, T. et al. Development of a miniaturized 96-Transwell air-liquid interface human small airway epithelial model. Sci. Rep. 10, 13022 (2020).
[6]. Blume, C. et al. Cellular crosstalk between airway epithelial and endothelial cells regulates barrier functions during exposure to double-stranded RNA. Immun. Inflamm. Dis. 5, 45-56 (2017).
[7]. Maniatis, N. A., Kotanidou, A., Catravas, J. D. & Orfanos, S. E. Endothelial pathomechanisms in acute lung injury. Vascul. Pharmacol. 49, 119-133 (2008).
[8]. Janga, H. et al. Site-specific and endothelial-mediated dysfunction of the alveolar-capillary barrier in response to lipopolysaccharides. J. Cell. Mol. Med. 22, 982-998 (2018).
[9]. Chowdhury, F., Howat, W. J., Phillips, G. J. & Lackie, P. M. Interactions between endothelial cells and epithelial cells in a combined cell model of airway mucosa: effects on tight junction permeability. Exp. Lung Res. 36, 1-11 (2010).
[10]. Papazian, D., Würtzen, P. A. & Hansen, S. W. K. Polarized Airway Epithelial Models for Immunological Co-Culture Studies. Int. Arch. Allergy Immunol. 170, 1-21 (2016).
[11]. Hermanns, M. I., Unger, R. E., Kehe, K., Peters, K. & Kirkpatrick, C. J. Lung epithelial cell lines in co-culture with human pulmonary microvascular endothelial cells: development of an alveolo-capillary barrier in vitro. Lab. Invest. 84, 736 (2004).
[12]. Hulme, K. D. et al. High glucose levels increase influenza-associated damage to the pulmonary epithelial-endothelial barrier. eLife 9, e56907 (2020).
[13]. Short, K. R. et al. Influenza virus damages the alveolar barrier by disrupting epithelial cell tight junctions. Eur. Respir. J. 47, 954-966 (2016).
[14]. Hermanns Maria Iris et al. An impaired alveolar-capillary barrier in vitro: effect of proinflammatory cytokines and consequences on nanocarrier interaction. J. R. Soc. Interface 7, S41-S54 (2010).
[15]. Salomon, J. J. et al. The Cell Line NCl-H441 Is a Useful in Vitro Model for Transport Studies of Human Distal Lung Epithelial Barrier. Mol. Pharm. 11, 995-1006 (2014).
[16]. Costa, A., de Souza Carvalho-Wodarz, C., Seabra, V., Sarmento, B. & Lehr, C.-M. Triple co-culture of human alveolar epithelium, endothelium and macrophages for studying the interaction of nanocarriers with the air-blood barrier. Acta Biomater. 91, 235-247 (2019).
[17]. Mul, F. P. J. et al. Sequential migration of neutrophils across monolayers of endothelial and epithelial cells. J. Leukoc. Biol. 68, 529-537 (2000).
[18]. Willems, C. H. M. P. et al. Alveolocapillary model system to study alveolar re-epithelialization. Exp. Cell Res. 319, 64-74 (2013).
[19]. Nishiguchi, A., Singh, S., Wessling, M., Kirkpatrick, C. J. & Möller, M. Basement Membrane Mimics of Biofunctionalized Nanofibers for a Bipolar-Cultured Human Primary Alveolar-Capillary Barrier Model. Biomacromolecules 18, 719-727 (2017).
[20]. Bhattacharya, R. F. H., Sunita Bhattacharya, Jahar. Crosstalk signaling between alveoli and capillaries—Rebecca F. Hough, Sunita Bhattacharya, Jahar Bhattacharya, 2018. Pulm. Circ. (2018).
[21]. Fischer, S. et al. Signaling mechanism of extracellular RNA in endothelial cells. FASEB J. 23, 2100-2109 (2009).
[22]. Frampton, J. P. et al. Rapid Self-Assembly of Macroscale Tissue Constructs at Biphasic Aqueous Interfaces. Adv. Funct. Mater. 25, 1694-1699 (2015).
[23]. Han, C., Takayama, S. & Park, J. Formation and manipulation of cell spheroids using a density adjusted PEG/DEX aqueous two phase system. Sci. Rep. 5, 11891 (2015).
[24]. Chandrasekaran, A. et al. Magnetic microboats for floating, stiffness tunable, air-liquid interface epithelial cultures. Lab. Chip 19, 2786-2798 (2019).
[25]. Lundholt, B. K., Scudder, K. M. & Pagliaro, L. A Simple Technique for Reducing Edge Effect in Cell-Based Assays. J. Biomol. Screen. 8, 566-570 (2003).
[26]. Walzl, A. et al. A Simple and Cost Efficient Method to Avoid Unequal Evaporation in Cellular Screening Assays, Which Restores Cellular Metabolic Activity.
[27]. Wagener, J. & Plennevaux, C. Eppendorf 96-Well Cell Culture Plate—A simple method of minimizing the edge effect in cellbased assays. 6.

1. A method of seeding cells to an underside of a surface, said method comprising:
providing a surface, wherein said surface is in an upright position;
providing a mixture that comprises cells and a cell suspension medium, wherein said cell suspension medium has a higher density than the cells; and
contacting the mixture with an underside of said surface thereby seeding the underside of the surface with said cells.

2. The method of claim 1, where the cell suspension medium comprises one or more of:
a polymer or mixture of polymers;
a small molecule or mixture of small molecules;
a synthetic or naturally derived particle or mixture of particles;
a coacervate;
a colloid;
a protein or mixture thereof;
a hydrogel or preparation thereof; and
a carrier solution selected from the group consisting of water, a buffer, a salt solution, and a cell culture medium.

3.-4. (canceled)

5. The method of claim 1, wherein the cells are epithelial cells.

6. The method of any claim 1, wherein the cells are on the surface of the cell suspension medium.

7. A method of creating a cell barrier, said method comprising:
providing a substrate in an upright position, wherein said substrate having a first surface and a second surface, wherein the second surface is located at an underside of the substrate;
providing a first mixture of cells that comprises a plurality of adherent cells and a cell suspension medium, wherein said cell suspension medium has a higher density than the adherent cells; and
contacting the first mixture of cells with the second surface, thereby seeding the underside of the substrate with the adherent cells.
adding a second mixture of cells that comprises a plurality of cells and a cell culture medium on the first surface of the substrate;
removing the cell suspension medium to form a mixed cell culture having an air and liquid interface whereby the adherent cells seeded on the underside of the substrate are exposed to the air, and whereby the cells on the first surface of the substrate are in contact with a cell culture medium, and
culturing the cells on the first and second surface at the air and liquid interface to form a cell barrier, wherein the cells on the first surface form a barrier on the first surface of the substrate, and the adherent cells on the second surface form a barrier on the second surface of the substrate.

8. The method of claim 7, wherein the substrate is porous between the first surface and the second surface.

9. The method of claim 7, wherein the substrate is coated with a polymer, a protein, a peptide, or a small molecule.

10. The method of claim 7, where the cell suspension medium comprises one or more of:
a polymer or mixture of polymers;
a small molecule or mixture of small molecules;
a synthetic or naturally derived particle or a mixture of particles;
a coacervate;
a colloid;
a protein or mixture thereof;
a hydrogel or preparation thereof;
a carrier solution selected from the group consisting of water, a buffer, a salt solution, and a cell culture medium; and
a dextran solution, a density gradient medium solution, or a combination thereof.

11.-14. (canceled)

15. The method of claim 7, further comprising exposing the seeded adherent cells to the air for at least 5 days.

16. (canceled)

17. The method of claim 7, wherein the cell barrier has an improved barrier function relative to a reference control.

18. The method of claim 17, wherein the improved barrier function is a measurement of trans-barrier electrical resistance of at least about 100 Ω·cm².

19.-23. (canceled)

24. The method of claim 7, wherein the adherent cells include H441 club cell line, engineered epithelial cells, or primary epithelial cells.

25. (canceled)

26. An in vitro tool for screening for or evaluating active agents that modulate a cell barrier or diagnosing a disease, wherein the in vitro tool comprises
a porous substrate configurable to an upright position,
an endothelial barrier, and
an epithelial barrier,
wherein the endothelial barrier is formed on a first surface of the substrate and the epithelial barrier is formed on a second surface of the substrate, wherein the second surface is located at an underside of the substrate.

27. The in vitro tool of claim 26, wherein said in vitro tool is prepared by:
providing a substrate in an upright position, wherein said substrate having a first surface and a second surface, wherein the second surface is located at an underside of the substrate;
providing a first mixture of cells that comprises a plurality of adherent cells and a cell suspension medium, wherein said cell suspension medium has a higher density than the adherent cells; and
contacting the first mixture of cells with the second surface, thereby seeding the underside of the substrate with the adherent cells.
adding a second mixture of cells that comprises a plurality of cells and a cell culture medium on the first surface of the substrate;
removing the cell suspension medium to form a mixed cell culture having an air and liquid interface whereby the adherent cells seeded on the underside of the substrate are exposed to the air, and whereby the cells on the first surface of the substrate are in contact with a cell culture medium, and
culturing the cells on the first and second surface at the air and liquid interface to form a cell barrier, wherein the cells on the first surface form a barrier on the first surface of the substrate, and the adherent cells on the second surface form a barrier on the second surface of the substrate.

28. An apparatus for screening for or evaluating active agents that modulate a cell barrier, wherein said apparatus comprises the in vitro tool of claim 25 and a multiple-well plate, wherein the in vitro tool is placed in or is a part of a well of the multiple-well plate.

29. (canceled)

30. A method of screening for or evaluating active agents that modulate an epithelial barrier or diagnosing a disease, said method comprising:
a) providing an apparatus of claim 15;
b) contacting the barrier on the first surface and/or the barrier on the second surface of the in vitro tool with an active agent;
c) contacting the barrier on the first surface of the in vitro tool with a plurality of cells,
d) contacting the barrier on the second surface with a fluid sample; and
e) measuring the number of the cells transmigrating to the side of the barrier on the second surface and/or determining a barrier function;
wherein an indication that the active agent impairs or improves the cell barrier includes at least one of a change in the number of the cells transmigrating to the side of the barrier on the second surface or a change in the barrier function.

31. The method of claim 30, wherein an increase in the number of the cells transmigrating to the side of the barrier on the second surface or a decrease in the barrier function is an indication that the active agent impairs the cell barrier, and wherein a decrease in the number of the cells transmigrating to the side of the barrier on the second surface or an increase in the barrier function is an indication that the active agent improves the cell barrier.

32.-35. (canceled)

36. The method of claim 30, wherein the active agent includes a chemical compound, a molecule, a toxin, or an organism.

37. The method of claim 30, wherein the fluid sample comprises a chemoattractant, a pro-inflammatory substance, or biological fluids obtained from a healthy subject or a patient having a disease.

38.-40. (canceled)

41. The method of claim 30, wherein the disease is a lung disorder includes chronic obstructive pulmonary disease, acute respiratory distress syndrome (ARDS), cystic fibrosis (CF), community acquired pneumonia, acute hypersensitivity pneumonitis, asthma, acute or chronic eosinophilic pneumonia, respiratory bronchiolitis, an influenza virus-associated lung disorder, a coronavirus-associated disorder, or a combination thereof.
